(19) United States
(12) Patent Application Publication
(10) Pub. No.: US 2025/0367024 A1
(43) Pub. Date: Dec. 4, 2025

(54) SYSTEM AND METHOD FOR IMPROVED THERMOTHERAPY

(71) Applicant: HYPERICE IP SUBCO, LLC, Irvine, CA (US)

(72) Inventors: Anthony Katz, Laguna Nigel, CA (US); John Parker Northrup, Hanson, MA (US); Robert Glen Edwards, San Diego, CA (US); Trevor Austin Kerth, San Diego, CA (US); Alexander Joseph Aguiar, San Diego, CA (US)

(21) Appl. No.: 19/227,127

(22) Filed: Jun. 3, 2025

Related U.S. Application Data
(60) Provisional application No. 63/655,168, filed on Jun. 3, 2024.
(60) Provisional application No. 63/680,886, filed on Aug. 8, 2024.
(60) Provisional application No. 63/719,872, filed on Nov. 13, 2024.
(60) Provisional application No. 63/767,279, filed on Mar. 5, 2025.

(51) Int. Cl. A61F 7/00; G16H 40/63

(52) U.S. Cl. CPC A61F 7/007; G16H 40/63; A61F 2007/0045; A61F 2007/0071; A61F 2007/0078; A61F 2007/0091; A61F 2007/0096

(57) ABSTRACT

A wearable thermotherapy apparatus includes at least one air bladder configured to conform to a contour of a portion of a user's body, at least one thermal element disposed adjacent to the air bladder and configured to apply heat to a targeted portion of the user's body, a support structure configured to limit outward expansion of the air bladder, a control unit configured to regulate heat and compression delivered via the thermal element and air bladder, a power source operably connected to the thermal element and control unit, and a user interface including one or more buttons and indicators to control and display compression and heat levels.

### CROSS-REFERENCE TO RELATED APPLICATIONS

This application claims the benefit of and priority to U.S. Provisional Patent Application No. 63/655,168 filed on Jun. 3, 2024, U.S. Provisional Patent Application No. 63/680,886 filed on Aug. 8, 2024, U.S. Provisional Patent Application No. 63/719,872 filed on Nov. 13, 2024, and U.S. Provisional Patent Application No. 63/767,279 filed on Mar. 5, 2025, the entire disclosure of each of which is hereby incorporated by reference in its entirety for all purposes.

### TECHNICAL FIELD

The present disclosure is directed to the field of therapeutic devices, and, more particularly, is directed to the field of devices that provide thermal and compression to selected portions of a body.

### BACKGROUND

Thermotherapy consists of the application of heat for the purpose of changing the cutaneous, intra-articular, and core temperature of soft tissue with the intention of improving the symptoms of certain conditions. Thermotherapy is a useful adjunct for the treatment of musculoskeletal injuries and soft tissue injuries. Using heat as a therapeutic intervention decreases pain in joints and muscles as well as soft tissues, and heat when applied properly, has certain effects on tissue metabolism, blood flow, inflammation, edema, and connective tissue extensibility. Various devices and facilities have been used to provide heat to the human body for use in rehabilitation facilities or at home. Exemplary devices include but are not limited to hot packs, wax baths, saunas, heat wraps, and steam baths/rooms.

One of the main problems found in these devices is that they become ineffective after a period of use and must be decommissioned, resulting in a short lifespan and limited reusability. This inefficiency makes them inconvenient for users who need continuous or long-term therapy. Additionally, these devices are often difficult to apply to body parts with irregular shapes, such as the heel or foot, because they cannot conform well to these areas. Even if they are carefully wrapped around the foot, gaps often form when the user moves, reducing the effectiveness of heat delivery.

Another major issue is that most existing devices are designed for stationary use. Facilities like saunas, wax baths, or steam rooms require the user to remain in one place for the duration of the therapy, which can be restrictive and impractical for people who need to remain mobile. While some wraps and hot packs are more flexible, they still suffer from issues like poor heat retention, limited application areas, and inadequate heat delivery to deeper tissue layers. These limitations reduce their therapeutic value, particularly when dynamic or active use is desired.

In addition, these existing devices generally do not integrate both heat and compression into a single, wearable unit. This separation means that while heat can be applied, it doesn't benefit from the increased tissue penetration that compression can provide. Without the compression element, heat therapy remains superficial and less effective for deeper tissue relief. Even in cases where heat and compression are combined, they often lack structural integration, causing the heating element to tear or separate from the compression component during use.

Furthermore, the existing thermotherapy devices offer little adaptability to different body shapes and sizes. They are usually designed for general use, without taking into account the unique anatomy of individuals. This one-size-fits-all approach results in discomfort and inefficient therapy for many users. Existing devices also lack user-friendly controls and real-time feedback, which limits the ability of users to adjust their treatment based on personal comfort and therapeutic goals. All these issues combined create a significant gap in the market for a device that can provide consistent, deep, and customizable heat and compression therapy in a convenient, wearable form.

Therefore, there is a need for an improved thermotherapy device that facilitates the flow of heat into the human body, no matter what the shape of the parts of the human body.

### SUMMARY

To address the aforementioned shortcomings, a method and system for improved thermotherapy is provided.

In one aspect, a wearable thermotherapy apparatus is provided in the disclosure, and the wearable thermotherapy apparatus includes at least one air bladder configured to conform to a contour of a portion of a user's body, at least one thermal element disposed adjacent to the air bladder and configured to apply heat to a targeted portion of the user's body, a support structure configured to limit outward expansion of the air bladder, a control unit configured to regulate heat and compression delivered via the thermal element and air bladder, a power source operably connected to the thermal element and control unit, and a user interface including one or more buttons and indicators to control and display compression and heat levels.

In some embodiments, the thermal element includes resistance wires disposed between two fabric layers. In some embodiments, the thermal element is integrated into the air bladder through adhesive bonding or stitching. In some embodiments, the apparatus further includes a heel counter or toe box as part of the support structure to prevent over-expansion of the bladder. In some embodiments, the air bladder includes a cavity that houses at least part of the thermal element.

In some embodiments, the control unit is embedded within a heel clip and includes a printed circuit board, a power regulator, a pump, solenoid valves, and a wireless communication component. In some embodiments, the power source is disposed within a midsole cavity and includes a rechargeable battery connected via a concealed tunnel. In some embodiments, the apparatus further includes a pressure sensor configured to detect user movement or therapeutic pressure and automatically adjust inflation levels. In some embodiments, the apparatus further includes a modular pod detachably coupled to the apparatus, housing the control unit and the power source.

In some embodiments, the wearable device is a shoe, sandal, slipper, jogger, vest, or glove. In some embodiments, the user interface includes a power button, heat and compression level buttons, and LED indicators showing power levels, heat levels and compression levels. In some embodiments, the thermal element includes a thermistor and thermal cutoff switch to prevent overheating.

In some embodiments, the air bladder includes a spandex-based lower structure and a neoprene upper support structure. In some embodiments, multiple zones of heat and compression are independently controllable within a single wearable device. In some embodiments, the thermal element includes a pair of resistance wires arranged in a symmetrical maze-like pattern between two fabric layers. In some embodiments, the thermal element is equipped with a heat spreader for improving thermal distribution across the contact area.

In another aspect, a method for delivering targeted heat and compression therapy to a user's foot is provided, and the method includes applying a wearable device including a thermal element and an inflatable bladder to the foot, selectively activating the thermal element to apply heat to a target region of the foot, selectively inflating the bladder to apply pressure to a same or overlapping region, dynamically adjusting the heat or pressure in response to user inputs or sensor feedback, and deflating the bladder or lowering heat upon detecting user motion or a preset condition.

In another aspect, a sandal for delivering heat and compression therapy to a user's foot is provided, and the sandal includes a sole including a midsole and an outsole, the midsole defining a housing for a power source and control electronics; an upper strap configured to secure the sandal to a user's foot, the upper strap including at least one thermal element configured to apply heat to a targeted portion of the foot, and at least one air bladder configured to apply compression to the same or overlapping portion of the foot; a control unit operatively connected to the thermal element and the air bladder, the control unit including at least one pump for inflating the air bladder; and a power source configured to power the thermal element and control unit, where the power source is housed within the midsole, and the thermal element and air bladder are integrated into the upper strap such that therapeutic heat and pressure are applied directly to a dorsal surface of the foot.

In some embodiments, the control unit further includes a temperature sensor and a pressure sensor for regulating heat and compression levels, and a user interface comprising one or more buttons and indicators integrated into the sandal. In some embodiments, the upper strap includes a removably attached pod, the pod housing the control unit and power source.

The above and other preferred features, including various novel details of implementation and combination of elements, will now be more particularly described with reference to the accompanying drawings and pointed out in the claims. It will be understood that the particular methods and apparatuses are shown by way of illustration only and not as limitations. As will be understood by those skilled in the art, the principles and features explained herein may be employed in various and numerous embodiments.

### BRIEF DESCRIPTION OF THE DRAWINGS

The foregoing aspects and other aspects of the disclosure are described in detail below in connection with the accompanying drawings in which:

FIG. 1 illustrates a cross-sectional view of a part of a heat and compression therapy apparatus, according to various embodiments;

FIGS. 2A-2B illustrate various views of an air bladder included in a heat and compression therapy apparatus, according to various embodiments;

FIG. 3 illustrates an exploded upper perspective view of a thermal element, according to various embodiments;

FIG. 4 illustrates a plan view of electrical heating wires on the lower sheet of the thermal element shown in FIG. 3;

FIG. 5 illustrates an exploded view showing an arrangement of thermal element components and an air bladder, according to various embodiments;

FIG. 6A illustrates an example attachment of a thermal element to an air bladder, according to various embodiments;

FIG. 6B illustrates an example attachment of a thermal element to a fabric sheet, according to various embodiments;

FIG. 6C illustrates an example heat and compression wrap, according to various embodiments;

FIGS. 7A-7C illustrate various external views of a heat and compression wrap, according to various embodiments;

FIGS. 8A-8C illustrate various internal and/or external views of a heat and compression wrap, according to various embodiments;

FIG. 9A illustrates an image showing a heel clip and heel counter configured for a heat and compression therapy apparatus, according to various embodiments;

FIG. 9B illustrates an interval view of a controller and other accessories inside a heel chip, according to various embodiments;

FIG. 9C illustrates various parts included in heel counter, according to various embodiments;

FIG. 9D illustrates a shoe prototype with an integrated heel clip and heel counter, according to various embodiments;

FIG. 10A illustrates perspective views of the heel clip and shoe midsole configured for a heat and compression therapy apparatus, according to various embodiments;

FIG. 10B illustrates an internal view of a midsole, according to various embodiments.

FIGS. 11A-11B illustrate example sandals with integrated heat and compression therapy wraps, according to various embodiments.

FIGS. 12A-12B illustrate an example plastic dock in a midsole for holding a battery pack, according to various embodiments;

FIG. 13 illustrates an example heated bladder footbed, according to various embodiments;

FIG. 14 illustrates an example block diagram of a heat and compression therapy apparatus, according to various embodiments;

FIG. 15 illustrates control buttons and associated status indicators for a heat and compression therapy apparatus, according to various embodiments;

FIG. 16 illustrates a flow chart of an example progress flow for a treatment session performed by a heat and compression therapy apparatus, according to various embodiments;

FIGS. 17A-17C illustrate prototypes of a jogger and a vest with integrated heat and compression therapy apparatus, according to various embodiments; and

FIG. 18 illustrates a block diagram of an example computer system, according to various embodiments.

### DETAILED DESCRIPTION

The figures (FIGS.) and the following description relate to some embodiments by way of illustration only. It is to be noted that from the following description, alternative embodiments of the structures and methods disclosed herein will be readily recognized as viable alternatives that may be employed without departing from the principles of the present disclosure.

Reference will now be made in detail to several embodiments, examples of which are illustrated in the accompanying figures. It is to be noted that wherever practicable similar or like reference numbers may be used in the figures and may indicate similar or like functionality. The figures depict embodiments of the disclosed system (or method) for illustration purposes only. One skilled in the art will readily recognize from the following description that alternative embodiments of the structures and methods illustrated herein may be employed without departing from the principles described herein.

As described earlier, the existing thermotherapy devices have many problems. The present disclosure addresses these problems and other problems in the existing thermotherapy devices by combining heat and compression into a single, highly adaptable device or system that is integrated into wearable structures like shoes or wraps. Unlike traditional wraps or hot packs that lose effectiveness over time and require the user to remain stationary, the thermotherapy device disclosed herein features air bladders and flexible heating elements that conform to the body's natural contours, ensuring consistent and effective contact even during movement. By integrating heating elements directly into a bladder system, the disclosed device ensures that heat is delivered uniformly to irregularly shaped body parts such as the heel or foot, which are difficult to treat using conventional hot packs or wraps.

One of the advantages of this system is the secure and flexible attachment of heating elements to the compression units. In typical devices, the heating element and compression layer are merely adjacent, which can cause tearing or separation when the compression unit inflates. The present disclosure resolves this by embedding the heating element within a bladder that can inflate and conform to the shape of the body part being treated. This integration prevents tearing and ensures that the heating element remains in close contact with the skin, maintaining efficient heat delivery.

Furthermore, the system's modular design allows it to be incorporated into wearable items like shoes, sandals, gloves, or even jackets. This transforms what was traditionally a stationary therapy into a portable and dynamic solution. Users can walk or move about freely while still receiving consistent heat and compression therapy, significantly expanding the context in which this therapy can be used, from home treatment to active recovery during sports. The air bladders in these wraps are adjustable, allowing them to dynamically fit different body shapes and sizes, addressing the problem of poor fit and uneven therapy coverage seen in traditional devices.

Additionally, the disclosed device or system includes sophisticated electronics and control systems that allow for real-time adjustments to heat and compression levels. Users can adjust the treatment intensity through control buttons or wireless connections to mobile devices, providing a personalized therapy experience that can adapt to their comfort and therapeutic needs. The system also incorporates sensors to monitor conditions like temperature and pressure, ensuring that the therapy stays within safe and effective ranges and further improving user comfort and safety.

By incorporating these improvements, the disclosed device and system transform heat and compression therapy into a dynamic, adaptable, and user-friendly solution that overcomes the traditional limitations of poor fit, limited motion, and inefficient heat delivery. It offers a much more convenient and effective way to deliver therapeutic heat and compression, significantly enhancing comfort and usability for a wide range of users.

It is to be noted that the benefits and advantages described herein are not all-inclusive, and many additional features and advantages will be further described under the context of specific embodiments. In addition, some additional features and advantages will become apparent to one of ordinary skill in the art in view of the figures and the following descriptions.

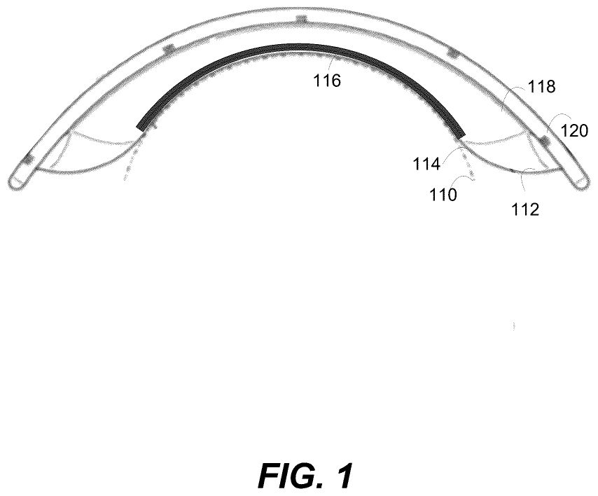

A heat and compression therapy apparatus 100 is illustrated in FIGS. 1-15. As described below, the heat and compression therapy apparatus can be applied to different locations of the body. For example, the heat and compression therapy apparatus 100 can apply heat to a selected location of the body, can apply compression to a selected location of the body, and can apply a combination of heat and compression to a selected location of the body. In one example, when applying heat to a selected location of the body, the compression can be selectively applied, to press the heat into the selected location of the body, thereby increasing the efficiency of heat delivery into the body. According to some embodiments, by providing compression while applying heat to a selected location of the body, heat may be delivered to a deeper part of the human body that may be not reachable without compression. For example, through combined heat and compression, a suitable amount of heat may be delivered to a layer of tissue adjacent to the bone, which converts the tissue at the bone layer from a gel-like to a more liquid viscosity. This can be accomplished by a combination of heat and pressure within certain bandwidth zones (e.g., high enough to achieve a therapeutic effect, but not so hot or intense as to hurt the wearer). The other existing thermotherapy devices cannot achieve such effects due to their limited functions of heat delivery into the human body.

According to some embodiments, the heat and compression therapy apparatus 100 can be adapted to be used with wraps, which can be flexibly tied to different portions of the human body that have different shapes and sizes, even for certain portions of the human body that are generally not feasible for the existing heat wraps or hot pads. In general, the concepts described herein can be applied to a wearable item wearable on any part of the human body. In one example, the heat and compression therapy apparatus 100 disclosed herein can be sewn into a shoe upper and wrapped around the heel and ankle to provide heat and/or compression to the heel and ankle area of a user, sewn into a shoe upper and wrapped around the toe area to provide heat and/or compression to the toe area of the user, and/or sewn into a sole of a shoe and wrapped around the foot bottom of a user to provide heat and/or compression to the foot bottom of user. FIG. 1 illustrates an example heat and compression therapy apparatus 100 that is flexibly wrapped or bent around a generally curved object 110 such as, for example, a human heel, limb, or joint (represented in dashed lines). The flexible bag-like inner structure 114 of a compression unit 112 (also referred to as “bladder”) readily conforms to the contours of the heel, toe, sole, limb, joint, etc. As illustrated in the figure, a thermal element 116 (i.e., heat generation element such as heat trace) is further disposed on the inner side 114 of the bag-like bladder facing the object 110. According to some embodiments, the thermal element 116 is integrated into the flexible bag-like inner structure of the compression unit 112 by directly adhering to the inner surface of the compression unit 112.

In some embodiments, the outer boundary 118 of the bladder can be supported by an additional support structure 120 that can hold the heat and compression therapy apparatus 100 against the object 110 receiving the therapy. In one example application for foot therapy, the outer boundary 118 of the heat and compression therapy apparatus 100 sewn into the heel/ankle area can be supported by a heel counter of a shoe, which itself is a semi-rigid structure in the heel area of a shoe that can provide support function, for example, by holding the heat and compression therapy apparatus without allowing the bag-like bladder to greatly expand during a heat and compression combined treatment. In another example application, the outer boundary of the heat and compression therapy apparatus 100 sewn into the toe area can be supported by a toc cap and/or toe box of a shoe, which itself is also a semi-rigid structure that can provide support function. In some exemplary applications, a bladder of the heat and compression therapy apparatus 100 would primarily inflate and hold at lower pressure so that it would not crush the toes of a user under the treatment. In yet another example, the outer boundary of the heat and compression therapy apparatus 100 sewn into the sole area can be supported by a sole of a shoe, which itself is also a semi-rigid structure that can provide support function. In some embodiments, by positioning the heat and compression therapy apparatus 100 in a structure (e.g., interleafed in the heel of a shoe) that includes a support unit (e.g., a heel counter), it may help maintain the integration between the thermal element (i.e., the heat generation unit) and compression unit (such as bag-like bladder) included in the heat and compression therapy apparatus 100. In the existing heat and compression devices, the thermal element and the compression unit are merely adjacent without direct coupling. Accordingly, when the compression unit is inflated, there is a risk that the thermal element can tear from the compassion unit due to a lack of flexibility of the thermal element. The heat and compression therapy apparatus 100 disclosed herein can overcome this problem by interleafing the integrated thermal element and compression unit such as a bag-like bladder into the heel area of a shoe upper. The shoe has a semi-rigid structure (e.g., heel counter) in the heel area that prevents the bladder from extending outward, thereby preventing tearing of the thermal element from the bladder. The expansion of the inner surface of the bladder is also limited by the foot of a user wearing the shoe, which also helps prevent the tearing of the thermal element from the bladder. In the next, the specific structures of the bladder and thermal element are further described.

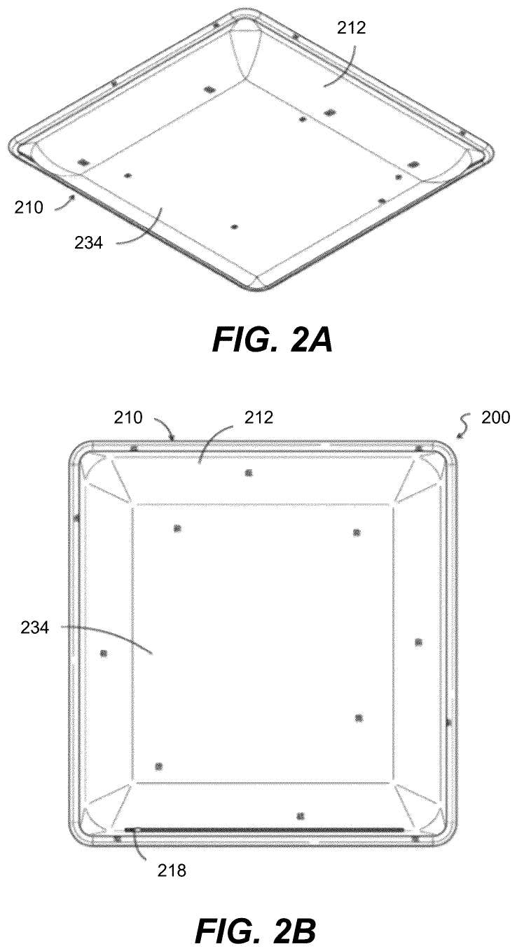

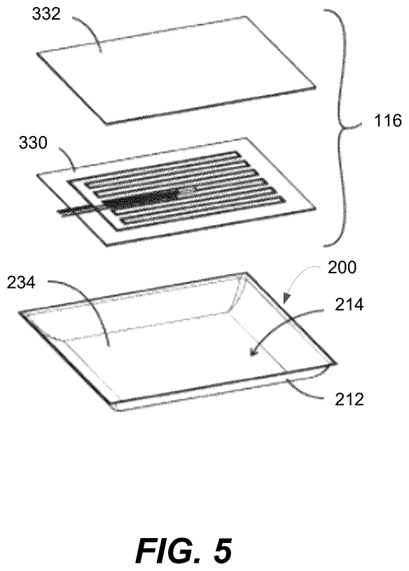

Referring to FIGS. 2A-2B, the structure of a bag-like bladder 200 is further described. As shown, in bladder 200 of the heat and compression therapy apparatus 100 is an enclosure 210. The enclosure 210 comprises a lower bag-like structure 212 that houses an inner cavity 214 (shown in FIG. 5). The lower bag-like structure 212 is secured to an upper support structure (not shown) and extends distally from the upper support structure. In the illustrated embodiment, the lower bag-like structure 212 comprises a strong elastomeric fabric such as, for example, a polyester-polyurethane copolymer fiber commonly referred to as “spandex.” In the illustrated embodiment, the upper support structure comprises a strong, flexible material. For example, the material may be an elastomeric material such as neoprene. Other strong materials can also be used instead.

In the illustrated embodiment, the lower structure 212 is sewn to the upper support structure along the four sides of the upper support structure. The seam between the two structures may be reinforced with bias tape or other material. In the illustrated embodiment, a zipper 218 is sewn into the lower structure to allow selective access to the cavity in the lower structure for the initial installation of certain components that can be included. The zipper 218 is positioned near one edge of the lower structure as shown. The zipper 218 is attached in such a manner that the edges of the fabric of the lower structure proximate to the two sides of the zipper are almost touching to substantially hide the underlying zipper from view. The material comprising the lower structure has generally rectangular dimensions sufficiently larger than the corresponding dimensions of the upper support structure such that the lower structure forms the inner cavity 214 with a sufficient depth relative to the upper support structure to accommodate certain components that may be included (e.g., at least one thermal element 116). In some embodiments, a cooling element (e.g., a thermoelectric cooler (TEC)) can be also included, so that both heating and cooling can be pressed to tissue via an inflated air bladder holding static pressure to achieve thermal compliance/efficiency.

In some embodiments, the upper support structure includes a through bore that is positioned close to the center of the upper support structure. The through bore has a sufficient size to accommodate a plurality of electronic wires (e.g., four, six, eight, ten, or twelve wires, etc.) for connection with a thermal element and/or a cooling element included in the cavity 214. In one example, the through bore may have a diameter between 0.1 inch and 0.25 inch.

In some embodiments, the bladder 200 may have just the lower bag-like structure 212 without necessarily having an upper support structure described above. For example, when there is only a thermal element without a cooling element, the bladder 200 may not have the upper support structure and a cavity to hold the thermal element. Instead, the thermal element 116 can be affixed to the bag-like bladder through an adhesive or other fixing mechanism without necessarily requiring a cavity to hold the thermal element.

As used herein, “bag-like structure” or “bladder” refers to various shapes the lower structure 212 may have when in use because the lower structure comprises a fabric material that is readily deformable to conform the material to irregular shapes. When the lower structure and the upper support structure are resting on a flat surface, the lower structure has a selected general shape defined by its outer dimensions such that a flexible distal (e.g., lowermost in the illustrated orientation) wall 234 of the lower structure is generally parallel to the upper support structure. The actual shape of the lower structure varies in response to the current shape of the upper support structure. For example, when the outer edges of the upper support structure are bent downward, the distal wall of the lower structure may sag away from the upper support structure. On the other hand, when the upper support structure is positioned behind a user's heel or other curved body part, the flexible distal wall of the lower structure can easily deform to conform to the irregular curvature of the body part.

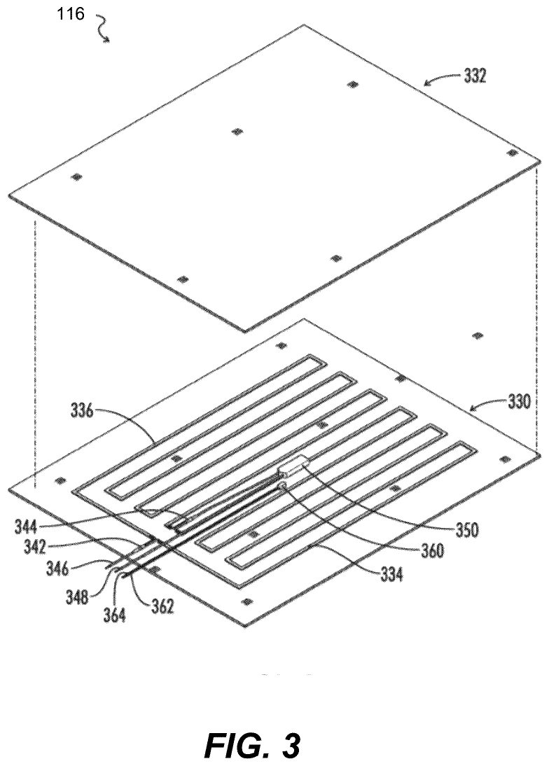

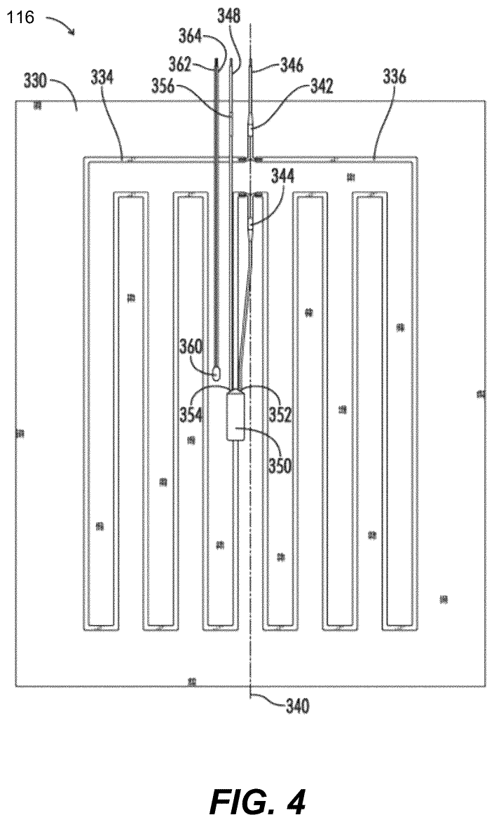

Referring now to FIGS. 3-4, the thermal element (or heat generation unit) 116 included in the heat and compression therapy apparatus 100 is further described. As illustrated in FIG. 3, in the illustrated embodiment, the heat generation unit 116 comprises a first (lower) rectangular sheet of cloth 330 and a second (upper) rectangular sheet of cloth 332. In the illustrated embodiment, each sheet comprises a 200 g needle punch material (i.e., non-woven material formed by a conventional needle punching process) having a thickness of approximately 1.5 millimeters. The material has a density of approximately 200 grams per square meter. At least one electrical resistance wire is positioned between the two sheets. In the illustrated embodiment, a first resistance wire 334 and a second resistance wire 336 are secured to the upper surface of the lower sheet 330 by lock stitching (not shown) in a conventional manner. The resistance wires can also be secured to the upper sheet in a similar manner. In one embodiment, each resistance wire comprises a thin, flat resistance wire, such as, for example, a commercially available titanium resistance wire, a nichrome wire, a copper trace, a carbon fiber element, or another type of resistance wire. In the illustrated embodiment, the cross-sectional dimensions of the resistance wires are selected to provide a resistance of approximately 16 ohms per meter or another proper value. In some embodiments, the thermal element 116 may be in other different shapes and structures and composed of different resistance materials for heating purposes, such as infrared heating elements, far-infrared heating elements, which is not limited in the disclosure.

As shown in FIGS. 3-4, the two resistance wires 334 and 336 form two maze-like patterns, which are substantially symmetric about a centerline 340 of the lower sheet 330. Each resistance wire extends from a first common terminal 342 to a second common terminal 344 such that the two segments are connected in parallel. The first common terminal of the resistance wires is connected directly to a first supply wire 346. The second common terminal of the resistance wires is connected to a second supply wire 348 via a thermal cutoff switch 350. The thermal cutoff switch has a first terminal 352 connected to the second common terminal of the resistance wires and has a second terminal 354 connected to the second supply wire via a connector 356. The thermal cutoff switch 350 is normally closed such that the control unit 140 is electrically connected to the second common terminal 344 of the resistance wires 334 and 336. The first common terminal 342 of the resistance wires is connected to the control unit. Thus, current is conducted from the first terminal around each of the first resistance wire and the second resistance wire in parallel. In one specific example, each resistance wire has a resistance of approximately 20 ohms, each resistance wire generates approximately 14 watts of heat at a voltage of approximately 16.8 volts, and the two resistance wires generate a total of approximately 28 watts of heat. In another example, the two resistance wires can generate another amount of heat.

The thermal cutoff switch 350 is set to open the circuit when the temperature proximate to the thermal cutoff switch exceeds approximately 80 degrees Celsius+/−5 degrees and stays open until the temperature reduces to approximately 55 degrees Celsius+/−10 degrees. In one embodiment, the thermal cutoff switch comprises a KLS-KSD9700 thermal fuse commercially available from Ningbo KLS Imp & Exp Co. Ltd. in Beilun of Ningbo China. The thermal cutoff switch is positioned across portions of the heating wire such that the thermal cutoff switch directly senses the temperature of the heating wire and disconnects the electrical path well before the heat from the heating wire is communicated through the lower sheet and the material of the lower structure 212 to a user (not shown).

As further shown in FIGS. 3-4, a thermistor 360 is secured to the first (lower) sheet of cloth 330. The thermistor is also positioned near the center of the first sheet; however, the thermistor is positioned between two adjacent segments of the first resistance wire 334 rather than directly on the resistance wire. A first wire 362 and a second wire 364 extend from the thermistor and are connected to the control unit 140. In one embodiment, the thermistor is a negative temperature coefficient (NTC) thermistor. For example, the thermistor may be an MF52-104F-3950-600L thermistor commercially available from Dongguan Xinxiang Electronic Technology Co., Ltd. in China. The thermistor has a resistance that varies over a wide temperature range. For example, at 55 degrees Celsius, the thermistor has a resistance of approximately 29,733 ohms; at 60 degrees Celsius, the thermistor has a resistance of approximately 24,753 ohms; and at 71 degrees Celsius, the thermistor has a resistance of approximately 16,794 ohms. The resistance of the thermistor is readily detectable in a conventional manner to determine when the temperature of the thermistor exceeds a selected temperature.

After the thermal cutoff switch 350 and the thermistor 360 are positioned on the first (lower) sheet 330, and after the first common terminal 342 is connected to the first supply wire 346 and the second common terminal 344 is connected to a second supply wire 348, the second (upper) sheet 332 is secured to the first sheet. In the illustrated embodiment, the lower surface of the second sheet includes an adhesive to removably attach the second sheet to the first sheet to form an integrated thermal element. Once the thermal element is formed, it can be attached to the bag-like air bladder 200 described in FIGS. 2-3.

Referring to FIG. 5, an exploded view shows how a thermal element 116 is aligned with an air bladder 200. In the figure, the first sheet 330 is shown to face the air bladder, while the second sheet 332 is on the side of the first sheet 330 away from the air bladder. However, the present disclosure is not limited to such configuration. In some embodiments, when the first sheet and the second sheet are attached to form a single piece of thermal element, the thermal element can be further attached to the air bladder from either side.

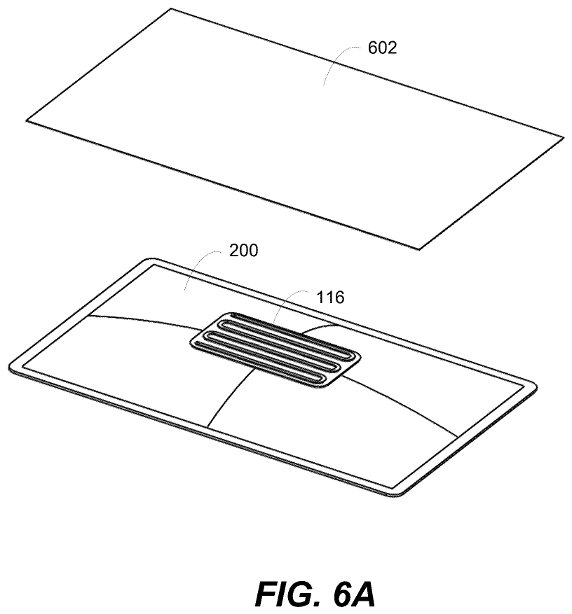

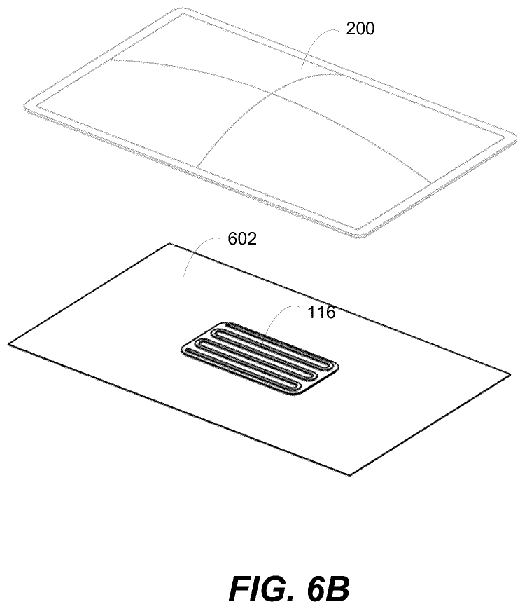

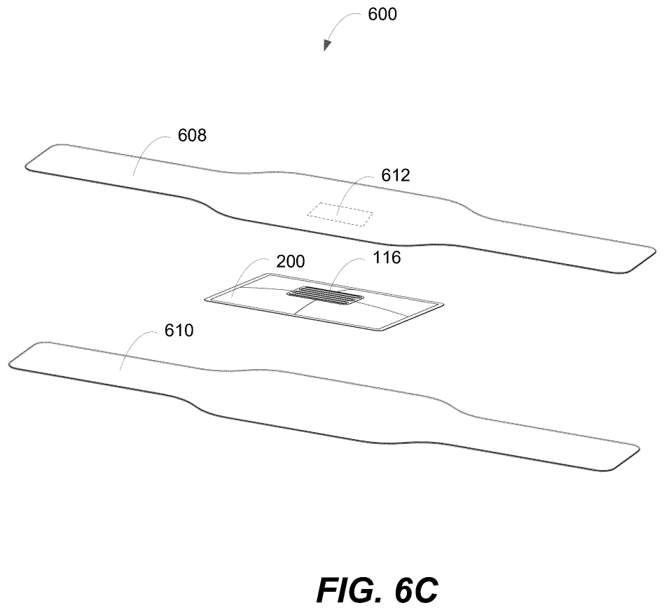

Referring to FIG. 6A, a thermal element 116 is shown to attach to an air bladder 200, according to some embodiment. In the illustrated figure, the thermal element 116 may correspond to the thermal element formed by attaching the first sheet and second sheet, as described earlier. In some embodiments, the thermal element 116 disclosed herein can further include a heat spreader (not shown) for facilitating heat delivery to the human body. In some embodiments, the heat spreader can attach the thermal element 116 from either side. In some embodiments, when the thermal element containing the heat spreader is further attached to the air bladder, such as air bladder 200 shown in FIG. 6A, the heat spreader is kept on a side away from the air bladder. In some embodiments, after the thermal element 606 and the air bladder 200 are integrated together, another fiber sheet 602 can be further provided to cover the integrated thermal element from a side away from the air bladder. In some embodiments, the fiber sheet 602 is part of the shoe upper, and thus the integrated thermal element and air bladder are sewn onto the fiber sheet 602.

Referring now to FIG. 6B, in some embodiments, instead of attaching to the air bladder 200, the thermal element 116 can attach to the fiber sheet 602 instead, as shown in the figure. Under such circumstances, the air bladder 200 and the thermal element 116 can be kept sufficiently close through the sewing process, for example, by wrapping the air bladder 200 and the thermal element 116 in a wrap, as further illustrated in FIG. 6C.

Referring now to FIG. 6C, an example heat and compression wrap 600 is further illustrated, according to some embodiments. As illustrated, the heat and compression wrap 600 includes a first sheet 608 and a second sheet 610 that wraps the air bladder 200 and thermal element 116 between the two sheets 608 and 610. In some embodiments, the first sheet 608 and/or the second sheet 610 can include a flexible fabric and/or an elastic material. In some embodiments, the first sheet 608 corresponds to the fiber sheet 602 shown in FIGS. 6A-6B. In some embodiments, the first sheet 608 can include one or more straps. In some embodiments, the second sheet 610 can also include one or more straps. In an example, the first sheet 608 and/or second sheet can include polyester and/or spandex. In some embodiments, the first sheet 608 and/or the second sheet can become an essential part of a shoe upper. In some embodiments, the first sheet 608 further includes one cavity 612 configured to receive and expose the heat spreader of the thermal element 606. In some embodiments, the first sheet 608 can be bonded to the second sheet 610. In an example, the first sheet 608 can be bonded to the second sheet 610 via sewing, stitching, gluing, and adhering, among other techniques and/or bonding processes. In an example, the first sheet 608 and the second sheet 610 can be sewn, stitched, glued, and/or adhered together with the thermal element 116 and the bladder 200 being kept between the two sheets. The whole structure can be referred to as a heat and compression wrap.

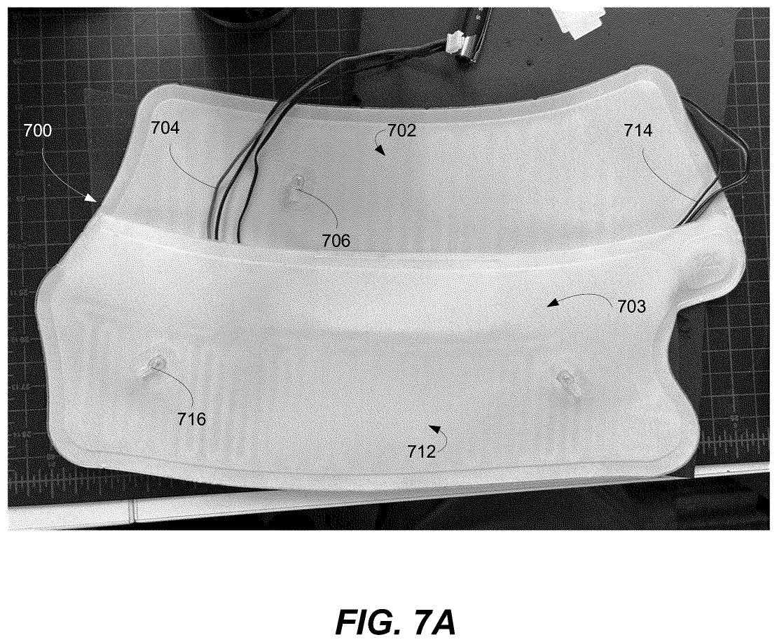

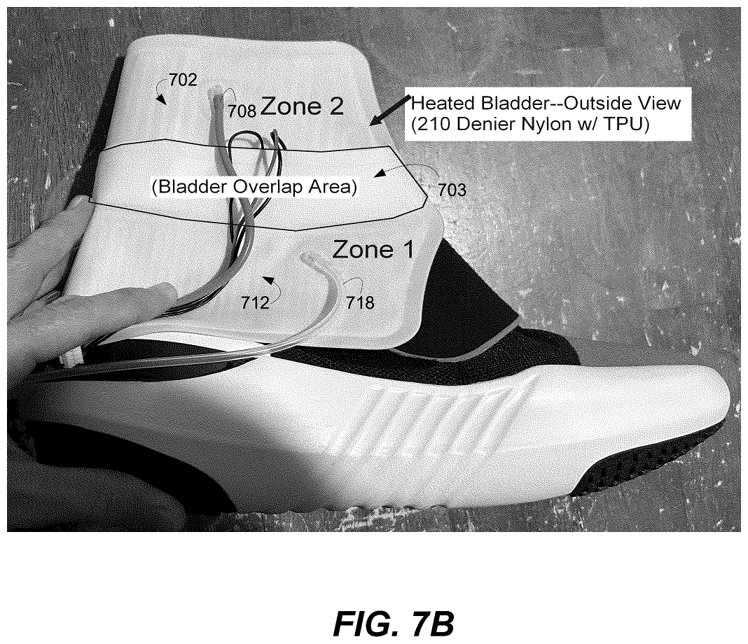

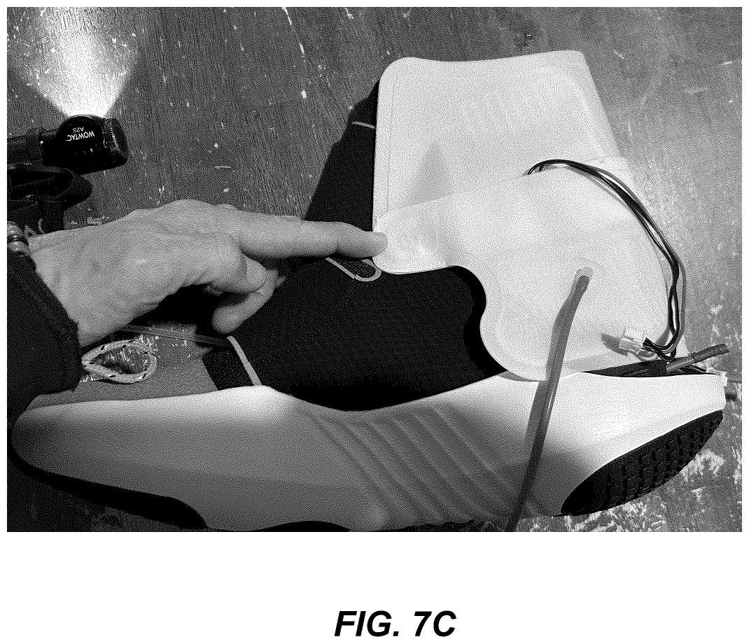

Referring to FIGS. 7A-7C, example external views of an actual heat and compression wrap 700 are shown, according to some embodiments. Specifically, FIG. 7A shows an example external review of a standalone heat and compression wrap 700 before being sewn into an actual shoe (more specifically a shoe upper), and FIGS. 7B-7C show example external reviews of a standalone heat and compression wrap 700 aligned in a way as if the heat and compression wrap 700 is sewn into a shoe (which it is not, since the heat and compression wrap 700 itself should be invisible after getting sewn into a shoe upper).

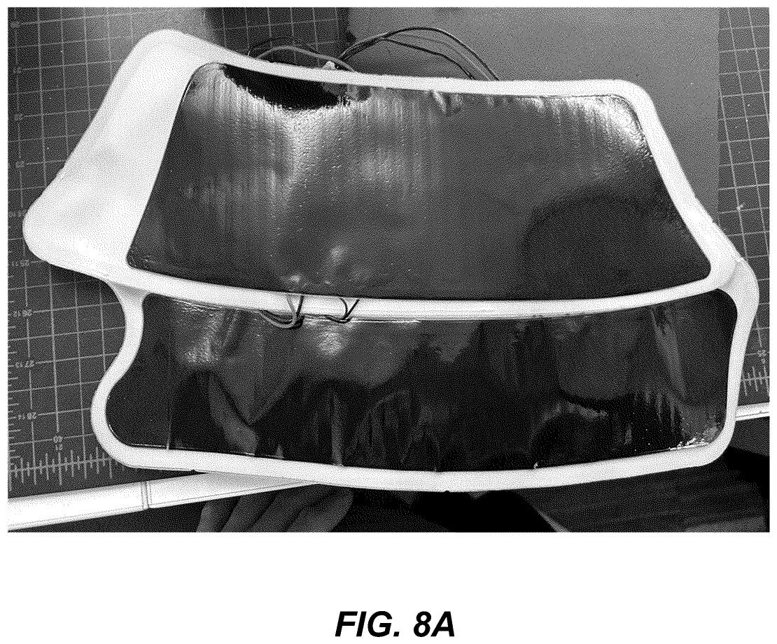

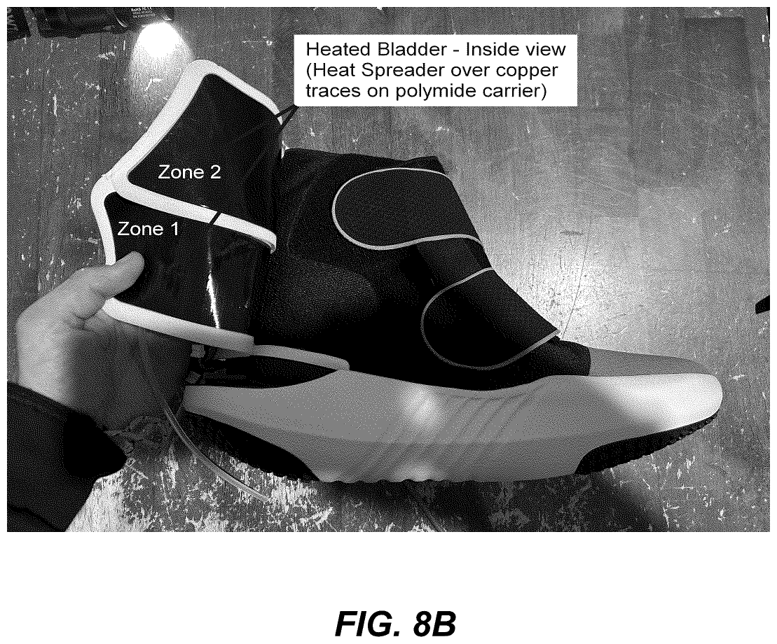

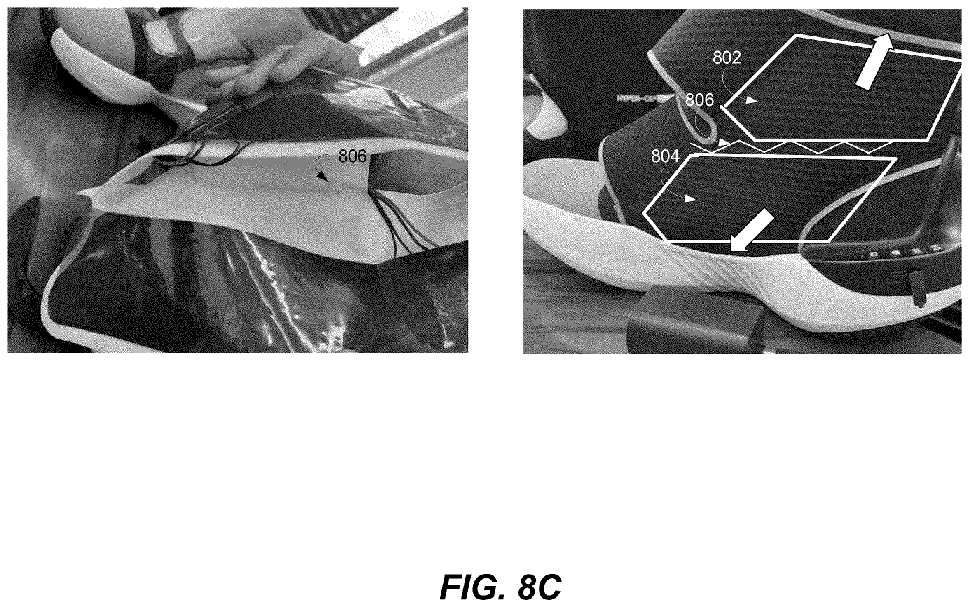

It is to be noted that “external” here means a distal side of a heat and compression wrap 700 away from the foot/heel of a person wearing the shoe after the wrap is sewn into the shoe upper. Correspondingly, “internal” means a proximal side of a heat and compression wrap 700 facing the foot/heel of a person wearing the shoe, as will be described in FIGS. 8A-7C.

Referring to FIG. 7A, a heat and compression wrap 700 can include one or more zones, each zone may correspond to a specific heat and compression wrap 600 shown in FIG. 6C. That is, each zone has its corresponding thermal element and air bladder, which can independently operate. In the embodiment shown in FIG. 7A, there are two zones 702 and 712 that are aligned side-by-side. Each zone 702 or 712 has a shape and size that is customized to match the shape and size of different portions of the shoe upper, as can be seen from FIGS. 7B-7C. According to some embodiments, by including more than one zone in a heat and compression wrap 700, it may allow different zones to have different heat and/or pressure during the treatment, which may save energy used by the whole heat and compression wrap 700, since there may be a situation where a special part may require more heat than the remaining part covered by the heat and compression wrap 700. This special part can be treated with a specific zone that can be independently operated, for example, by providing a higher level of heat and/or pressure.

It is to be noted that, in actual applications, depending on where a heat and compression wrap is intended to be used, there may be other possible numbers of zones, where each zone can have a shape and size that is customized to match the size and shape of different parts of a human body. In addition, as shown in the embodiment in FIG. 7A, when there are two (or more) zones in a heat and compression wrap 700, there can be also an overlap region 703 where edges (the outer section of a heat and compression wrap 600 that does not include a thermal element and/or air bladder) of the different zones meet. By allowing the edges of different zones to overlay, it can save the space taken by the edges, leaving more space for the remaining thermal element and/or air bladder. In some embodiments, by including the overlap region 703, it can increase the strength of the whole heat and compression wrap 700, allowing it to hold a higher pressure and prevent a thermal element from tearing from the respective bladder.

In some embodiments, not only different zones in a same heat and compression wrap 700 have different shapes/sizes, each zone between different heat and compression wraps 700 can also have different shapes or sizes, depending on the size of a shoe for a heat and compression wrap to be sewn into. In some embodiments, a predefined set of heat and compression wraps 700 with different shapes/sizes can be sewn into shoes of different sizes, to meet the different foot sizes among a large number of customers. In some embodiments, a heat and compression wrap 700 can be specifically customized in shape and size to meet a user's specific requirements.

In some embodiments, when including the disclosed heat and compression wraps into shoes of different sizes, these compression wraps can facilitate accommodating feet of a larger range of sizes when compared to other conventional shoes, since a heat and compression wrap(s) can flexibly tighten feet of different sizes through pumping different amounts of air into the compression wrap(s). Accordingly, when designing shoes with the disclosed heat and compression wrap(s), a smaller number of shoe sizes are necessary when compared to other conventional shoes. For example, for conventional shoes, a shoe company may prepare shoe sizes of 5, 5.5, 6, 6.5, 7, 7.5, 8, 8.5, 9, 9.5, 10, 10.5, 11, 11.5, 12, and so on for different customers. For the shoes with the disclosed heat and compression wraps, a much smaller number of shoe sizes are needed for different customers. For example, the needed shoe sizes may be just 5, 6, 7, 8, 9, 10, 11, 12, etc., or may be just 5, 6.5, 8, 9.5, 11, 12.5, etc., or may be just 5, 7, 9, 11, 13, etc., or may be just 5, 8, 11, etc. The exact number of shoe sizes and the exact sizes for these shoes may vary and depend on the size or volume of an air bladder in a heat and compression wrap. In addition, the exact number of heat and compression wraps sewed into/onto a shoe may also affect the exact number of shoe sizes required for the market or different customers. For example, if heat and compression wraps are also bottom and/or toe areas of shoes besides the upper cover areas, an even smaller number of shoe sizes may be required for marketing purposes.

In some embodiments, when the shoes with heat and compression wraps are provided to customers, these shoes may recognize different foot sizes of different customers through different means, and then provide necessary adjustments by pumping different amounts of air into air bladders to fill in the gap so as to provide proper comfortability even a user is not under a heat and compression treatment. In some embodiments, these shoes may include certain controllers as described elsewhere, which may facilitate understanding the foot size of a customer based on the information provided by the customer, e.g., received from mobile devices through wireless communication or through user inputs configured for certain types of shoes. Additionally, or alternatively, these shoes may have certain pressure sensors or other types of sensors that can feel or sense the foot sizes or customers. Once the foot size of a user is identified, it can be determined how much air should be pumped into the air bladder(s) of the heat and compression wrap(s) sewn into a shoe, even if the user is not under therapy or treatment. In some embodiments, when the user desires to get treatment or therapy, additional air and/or heat may be provided to the customer as described elsewhere herein.

Under certain circumstances, if the user wants to take off the shoe, the air pumped into the air bladder(s) can be released a little or totally released right before that, so that a user can easily take off the shoe when compared to other conventional shoes. In some embodiments, the air pumped into the air bladder(s) may be released after the user takes off the shoe instead. In some embodiments, the air may not be released at all even if the user does not wear the shoe anymore.

In some embodiments, the heat and compression wraps described above may be not necessarily purposed for the heat and compression treatment. Instead, these heat and compression wraps may be just compression wraps sewed into shoes, to increase the comfortability of customers and/or increase the accommodation range of these shoes, so that a customer does not need to try a shoe before purchasing. Under such circumstances, a simpler version of the heat and compression wrap described elsewhere may be used here, since a heating element may not be necessary for this purpose.

Referring again to FIG. 7A and as described earlier, each zone 702 or 712 of the heat and compression wrap 700 can include a thermal element (not obviously shown in the figure), a set of electrical wires 704 or 714 connected to the respective thermal element (e.g., heat trace), an air bladder, and one or more tube ports 706 or 716 for the respective air bladder. As will be described in detail later, each air bladder can be equipped with one or more tubes that are operatively connected to an air pump or compressor for inflating air to one or more compression pads included in the air bladder during the treatment. A tube can be detachably connected to a tube port 706/716 through screwing or through other locking mechanisms.

Referring to FIG. 7B, an example external review of a standalone heat and compression wrap 700 aligned in a way as if it is sewn into a shoe is illustrated. Two different zones 702 and 712 and an overlap region 703 are shown in the figure. Also shown in the figure are two tubes 708 and 718 that are connected to the tube ports in each of the two zones 702 and 712 shown in FIG. 7A. As also shown in FIG. 7B, the sheet(s) for wrapping the thermal element and air bladder can include polyester and/or spandex. In the illustrated embodiment, a 210 Denier nylon coated with a thermoplastic polyurethane (TPU) membrane is used. In other embodiments, other different materials can be used.

Referring to FIG. 7C, another example external review of the standalone heat and compression wrap 700 shown in FIG. 7B is illustrated. The external views shown in FIGS. 7B-7C are for two different sides (e.g. left and right sides) of the same heat and compression wrap 700. Similar to FIG. 7B, there are two different zones and an overlap area, a set of electrical wires, and a connected tube for the lower zone.

Referring now to FIGS. 8A-8C, example internal views of a heat and compression wrap 700 are shown, according to some embodiments. As mentioned earlier, the internal views mean an inner-side view of a heat and compression wrap 700 after being sewn into a shoe. FIG. 8A shows a standalone internal view and FIG. 8B shows an internal view as if the heat and compression wrap 700 is sewn into a shoe upper (which it does not).

As can be seen in FIG. 8A, compared to the external views, the internal view of a heat and compression wrap 700 shows a heat spreader that is exposed through a cavity in each zone. In some embodiments, when the heat and compression wrap 700 is sewn into a shoe, the heat spreader in each zone is left exposed, and thus the heat spreader in each zone can directly contact with the foot/heel of a user, which facilitates providing heat to the user wearing the shoe. FIG. 8B shows an interval view aligned with a shoe upper, where two heat spreaders are shown to be exposed to the user. In some embodiments, the heat and compression therapy apparatus 100 includes additional elements to control heat and compression delivery through the heat and compression wrap 700.

The left image in FIG. 8C shows another internal view of a heat and compression wrap 700 and right image in FIG. 8C shows another external view of a heat and compression wrap sewed to the show upper of a shoe. As can be seen in the right image, there are two different zones (e.g., zone 802 and zone 804) aligned up and down. When the air bladders get inflated in these two zones, the upper air bladder may be pushed up while the lower air bladder may be pushed down, as indicated by the two arrows in the image. This is mainly due to the more free space above or below these two zones. To prevent these two zones from moving too far away from the desired locations, an additional tethering part 806 may be sewed in the wrap, as can be seen in the left image (also indicated by the seesaw line 806 in the right image). This tethering part 806 may be made of material with very limited elasticity and with a high young modulus so that it can hold high pressure without much change in structure. This then allows the different zones to hold at the desired locations when the air bladders get inflated and/or deflated.

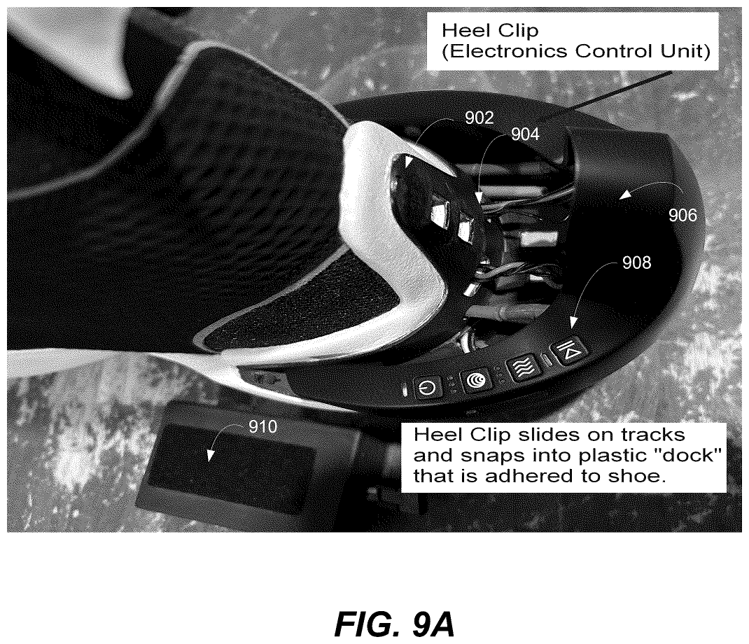

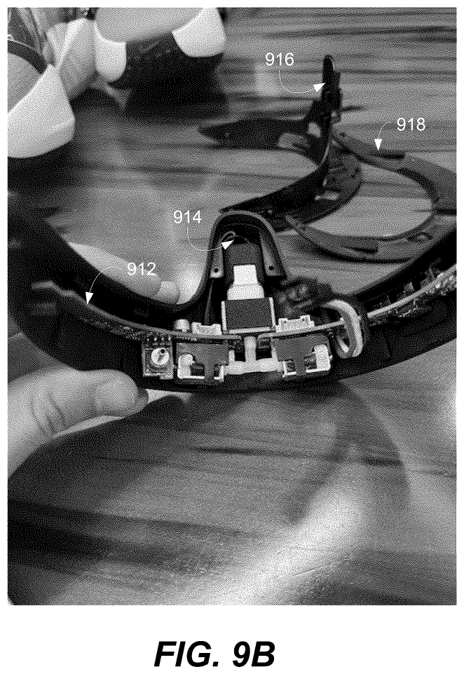

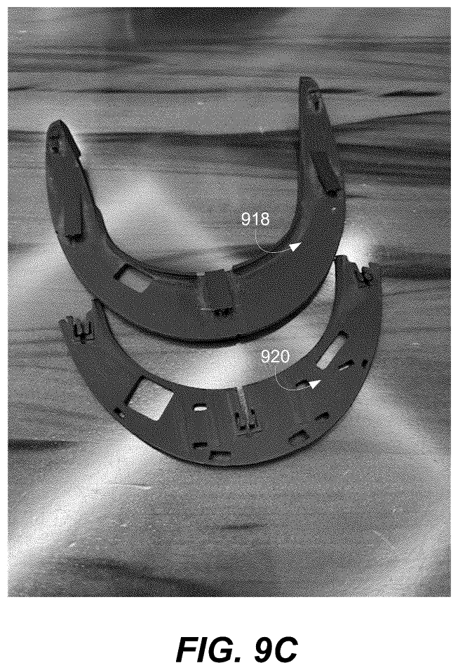

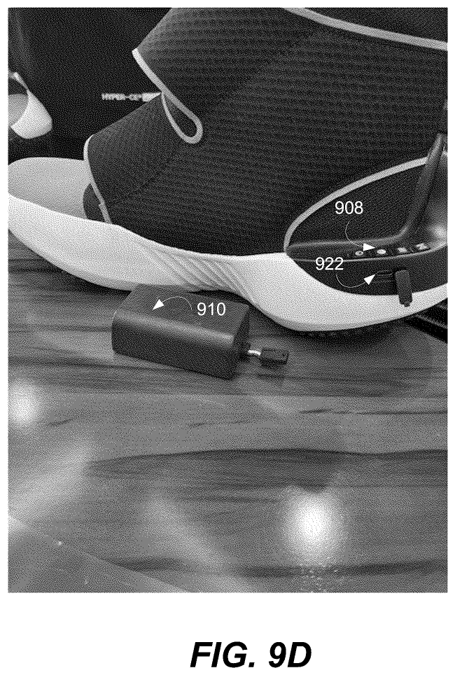

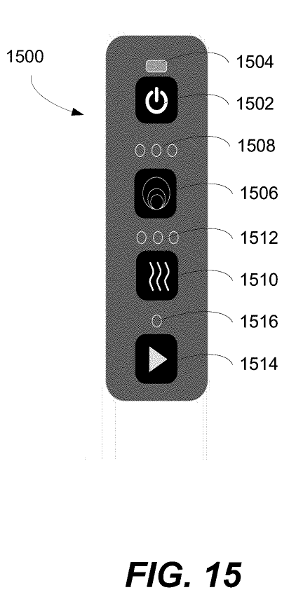

Referring now to FIG. 9A, the electronics control unit (ECU) and other accessories for the heat and compression apparatus 100 are further illustrated, according to some embodiments. The electronics control unit 906 disclosed herein is configured in a form like a heel clip and thus is also referred to as a “heel clip.” While not clearly shown, the heel clip 906 includes certain control units and other components for controlling the operation of the heat and compression wrap 700, as illustrated further in detail in FIG. 9B. In one example, the heel clip 906 includes a set of control buttons and corresponding indicators 908, as can be seen in the image in FIG. 9A. The specific functions of these control buttons and state indicators are further described in detail in FIG. 15.

As also shown in the image in FIG. 9A, the shoe has a semi-rigid structure 902 (e.g., heel counter) in the heel area that helps prevent the air bladder included in the heat and compression wrap from extending outward, thereby preventing tearing of the thermal element from the bladder. In addition, there are certain electronic wires that connect the electronics control unit 906 with the thermal element in different zones, as described earlier. Accordingly, there is at least one portion in the semi-rigid structure 902 that provides a channel for the electronic wires to pass through. In some embodiments, the semi-rigid structure 902 also includes one or more locking mechanisms for locking the heel clip 906 to the shoe, as further described in FIG. 9C. This allows a user wearing the shoe to continue the treatment while walking around with the shoe. That is, the user is not required to keep stationery during the treatment.

Referring now to FIG. 9B, an image showing certain control units and other components for controlling the operation of the heat and compression wrap is illustrated. As can be seen from the image, a printed circuit board (PCB) 912 is aligned along a curvature of the heel clip 906, which provides a medium used to connect or “wire” components to one another. Exemplary components that can be connected to the PCB 912 include but are not limited to the power supply, control button, heating element and electronic motor included in an air pumping element, sensors, and so on. The exact details of some components are not specifically described here and can refer to U.S. patent application Ser. Nos. 18/300,261 and 17/943,181, each of which is incorporated herein by reference in its entirety. As can be seen in the image, in some embodiments, there are one or more protrusions (one in the middle shown in FIG. 9B) to allow certain elements with a larger size (e.g., heating and/or pumping elements) to be fit into the heel clip.

Also shown in FIG. 9B is a prototype of a part of heel counter 916. As can be seen, the heel counter 916 may also have a curvature that includes an extended upper part for providing support as described above. The exact shape and size including its curvature may be configured to fit the shoe size as well as the heel clip 906 described above, so that when these various components are integrated together, these components can match with each other to provide a compact design.

In addition, tightening or fixing these various components together is also important in the design of the disclosed heat and compression wrap, since these shoes are designed also for mobile use but not just for stationary treatment. For this purpose, additional mechanisms or components for fixing or attaching heel clips and/or helping counter to a shoe are also contemplated.

FIG. 9C shows two pieces/elements 918 and 920 (the element 918 is also shown in FIG. 9B) for fixing purposes according to some embodiments. As can be seen, these different pieces/elements 918 and 920 include specifically configured holes/throughs/vias/slits/slots/concaves on one piece and certain matching blocks/protrusions on the other piece. These two fixing pieces, when placed together, allow them to lock to each other through these different parts. In some embodiments, before locking these two fixing pieces together, one piece (e.g., the fixing piece 918) may be movable and first positioned to hold the heel counter and/or heel clip tight, while other pieces (e.g., the fixing piece 920) may be fixed attached to the shoe (e.g., over the outer sole of the shoe). Once these two fixing pieces are locked together, the various elements can be locked to the desired positions, and generally do not move even if a user wearing the shoe takes some intensive actions (e.g., certain running or other activities).

It should be that the fixing pieces shown in FIGS. 9B-9C are for exemplary purposes, and not for limitations. There are certain other structures and components that may be able to provide tightening different components to a shoe. In addition, while not shown, there may be certain tools that can unlock or decouple these two fixing pieces 918 and 920, so that the interval components can be still accessible if necessary. For example, a battery may expect a replacement after a certain time of use, and a tubing may be broken due to an accident and need a replacement.

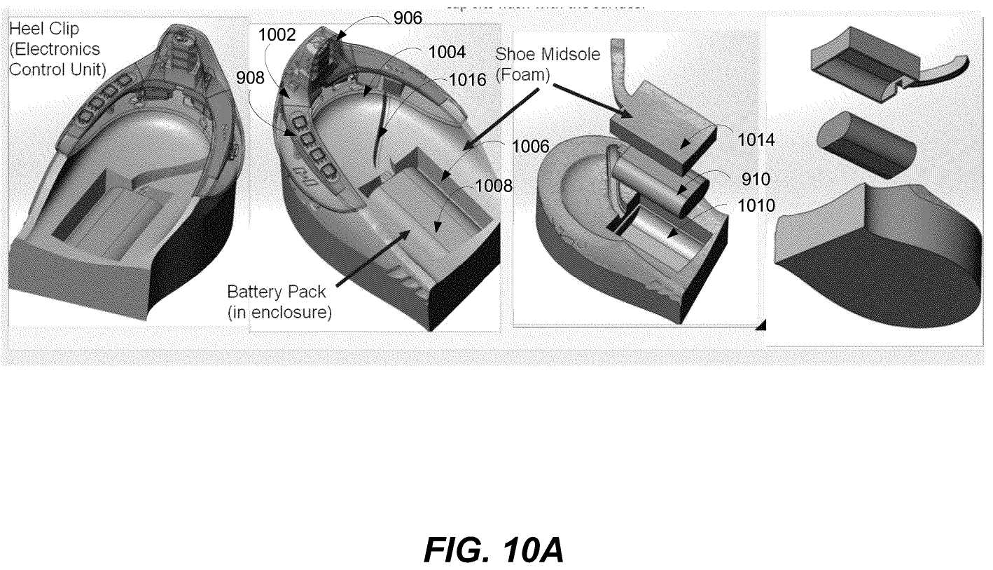

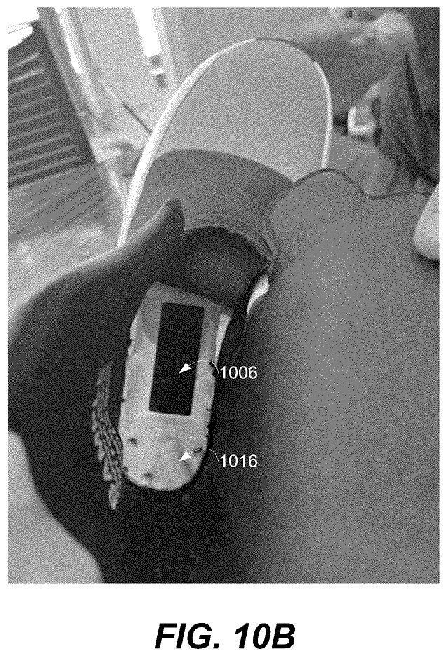

FIG. 9D shows an image of a shoe prototype with an integrated heel clip and heel counter, according to various embodiments. As can be seen in the image, once integrated, the various interval components inside the heel clip and/or heel counter are generally not visible. A user can control the operation through the buttons 908 as described earlier. As also shown in the image in FIG. 9D (also in FIG. 9A), the heat and compression therapy apparatus 100 also includes a battery 910 for providing power to the heat and compression therapy apparatus 100. In some embodiments, the battery 910 may be rechargeable, and accordingly, there is also a port 922 for charging the battery through a certain charger, such as an AC adaptor. It should be noted that, while the battery 910 is shown outside the shoe in FIG. 9D, in actual applications, the battery 910 is placed in the shoe midsole and is invisible to a user, as further described in detail in FIG. 10A.

Referring now to FIG. 10A, a series of views of a shoe midsole and a heel clip are further illustrated. As shown in the transparent views in the left two images, the heel clip 906 disclosed herein includes a printed circuit board (PCB) 1002 (which may be the same or similar unit as PCB 912 shown in FIG. 9B) and a set of buttons and associated LED indicators corresponding to the ones shown in FIG. 9A. While not shown in exact detail, the heel clip 906 also includes a compressor or air pump, and a set of valves for controlling air inflation and deflation. In addition, the heel clip 906 also includes a pressure sensor for sensing pressure provided to the air bladders. In some embodiments, the same pressure sensor or another different pressure sensor may be used to monitor pressure in a bladder sewn into the sole area of a shoe, which can be then used to determine whether the user is relaxing or moving. For example, if the pressure sensor feels a big spark within the bladder at the foot bottom, it indicates that the user may stand up and begin to move. At this point, the bladder sewn into the foot bottom area may no longer be inflated or get deflated if already inflated, to make sure no force/pressure is pushing from the bottom of the foot while the user is moving.

In some embodiments, there may be other different sensors that can be used for motion detection, which can be also included in the heat and compression therapy apparatus 100. In one example, an accelerometer may be disposed within the heel clip 906, where the accelerometer is used to detect whether the person is relaxing or moving. In some embodiments, a user may have a wearable device for motion detection, which may be configured to transmit the detected motion to the heat and compression therapy apparatus 100. Based on the detected motion, the heat and compression therapy apparatus 100 may also control the bladder in the foot bottom area deflated or not inflated when the user is moving.

In some embodiments, similar mechanisms may be also used to control a bladder sewn into other parts of a foot and/or other portions of a body. For example, if an accelerometer or wearable device detects that a user runs faster, a bladder sewn into a jacket, jogger, or vest also gets deflated, to make sure that these bladders do not interfere with the warmup of the user. In some embodiments, the bladders sewn into the ankle/heel area and/or toe area may be also deflated or at least adjusted for inflated pressure, if necessary, to make sure that the user is comfortable during his/her movements.

As also illustrated in FIG. 10A, the shoe midsole 1004 includes a housing unit 1006 for housing a battery pack 1008. The battery pack 1008 includes a battery holding chamber or enclosure 1010, a battery 910, and a battery cover 1014. The battery 910 may be a rechargeable battery, such as a LiPo battery, and may include one or more battery units that can be placed into the holding chamber 1010. On one side (e.g., the side facing the heel clip) of the battery holding chamber 1010, there is also a tunnel 1016 that provides space for the electronic wires that connect the battery pack 1008 to the components such as PCB included in the heel clip 906. By hiding the electronic wires in the tunnel 1016, the surface of the shoe midsole can remain generally flat for better user experience.

FIG. 10B further provides an internal view of the midsole included in a shoe prototype. As can be seen, there is a housing unit or chamber 1006 for housing a battery described above. Also included is a tunnel 1016 for holding electronic wires connecting the battery to the PCB and/or other components.

In some embodiments, the shoe disclosed herein is not limited to traditional walking, hiking, or sports shoe types, but can be any type of footwear. For example, the heat and compression therapy apparatus disclosed herein 100 can be incorporated into a ski boot, a fishing boot, a farm or ranch boot, or a slider/sandal/flip-flop that is malleable, or bendable or pliable 360-degree heat/compression soft shoe, or any other type of footwear not described above. In some embodiments, besides footwear, the heat and compression therapy apparatus 100 can be incorporated into a jacket, a jogger, a vest, or any other type of wear, which is not limited in the present disclosure.

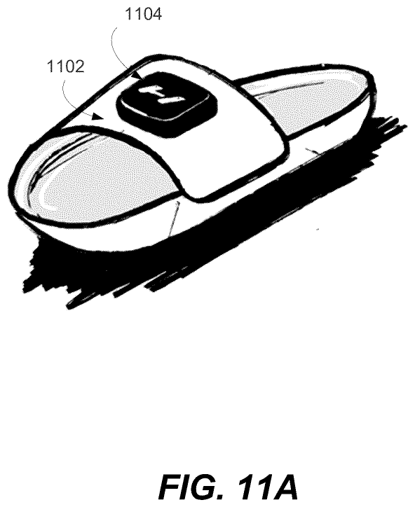

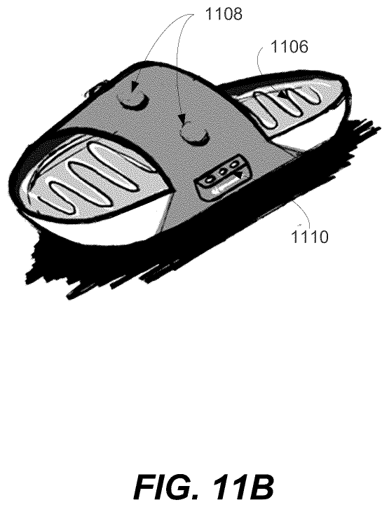

Referring now to FIGS. 11A-11B, the heat and compression therapy apparatus disclosed herein is integrated into a slider/sandal/flip-flop. In one specific example, a sandal may be upgraded with one or more sleek, integrated heat and compression therapy wraps. A wrap contains embedded heating elements to provide warmth, along with small compression nodes (also referred to as vibration nodes due to its vibration feature) that offer a gentle massage. In some embodiments, additional LEDs or other types of indicators can be further provided to indicate the adjustable heat and compression settings, which could be controlled via a small panel attached to the wrap, or through mobile devices communicatively coupled to the heat and compression therapy wrap. The sandal would keep its lightweight, flexible design, with an advanced twist for therapeutic comfort.

Specifically, referring to FIG. 11A, a heat and compression therapy wrap can be disposed along or integrated into an upper strap 1102 shown in the figure. The wrap includes embedded heating elements and may also include the compression nodes, which can be controlled by a controller integrated into the wrap. In some embodiments, more than one wrap can be integrated into a single sandal depending on the specific design, where each wrap may operate independently or collaboratively. In addition, each wrap may include its own power supply such as a battery (which can be rechargeable and/or replaceable), or all wraps integrated into a sandal are controlled and powered by a shared controller and a shared power supply. In some embodiments, the controller and/or power supply may be integrated into the midsole or outsole, so as to provide more space for integrating the heating elements and/or compression nodes into the straps. When integrated into the midsole or outsole, a charging port may be exposed for recharging the battery integrated therein. In some embodiments, the controller and/or power supply, when integrated into the midsole or outsole may provide wireless/wired charge and/or control signals to the heating elements or compression nodes. In some embodiments, the additional LEDs can be disposed on the surface of the footbed when the controller and/or power supply are integrated into the midsole or outside of a slider/sandal/flip-flop.

In some embodiments, the controller and power supply may be configured inside a pod, which can be removably attached to a heat and compression therapy wrap, as indicated by the pod 1104 in FIG. 11A. The detachable pod may be taken away from the slider/sandal/flip-flop for battery recharge or for other purposes. In some embodiments, the pod 1104 may be magnetically attached to a heat and compression therapy wrap. In some embodiments, the pod may be mechanically attached to a heat and compression therapy wrap, such as through snap-fit, press fit, rivet, cam lock, latch, buckle, press fit, or other attachment and quick-release mechanisms. In another example, to facilitate the intense activities experienced by a user, additional or alternative attaching or fixing mechanisms may be also utilized. For example, a small strap may be put over the pod to tighten/hold the pod more stably, where the pod may be directly attached to the heat and compression therapy wrap magnetically or non-magnetically. In some embodiments, the pod itself may be integrated into the midsole or outsole area, but not detachably attached to the heat and compression therapy wrap.

In some embodiments, the compression may be generated through an air pump configured inside the pod. In other embodiments, the pod may not include an air pump, where the air pump may be located inside another box fixedly attached to the heat and compression therapy wrap. In yet other embodiments, the heat and compression therapy wrap may not include a compression function but rather only include a thermal therapy function, to minimize the weight of the heat and compression therapy wrap, thereby facilitating the movements of the user wearing the slider/sandal/flip-flop.

In some embodiments, additional heating elements and/or compression nodes may be also integrated into the footbed (or sole area) of a slider/sandal/flip-flop, so that the foot bottom of a user may be also subjected to heat and compression therapy. The waves 1106 in FIG. 11B indicate the possible heat wrap integrated into the footbed or sole area. The arrows 1108 in FIG. 11B indicate the possible vibration nodes integrated onto the heat and compression therapy wrap. The arrow 1110 indicates the possible battery and/or controller box, which further includes certain LEDs or other types of indicators for indicating the adjustable heat and compression settings. All of these elements in FIGS. 11A-11B are provided for exemplary and illustrative purposes and not for limitations. The exact size of pods 1104 may vary depending on the specific power supply included therein, among other factors that may affect the eventual size of a pod. The specific details for the pod configuration may refer to the U.S. patent application Ser. No. 17/943,818, the entirety of which is incorporated herein by reference.

In some embodiments, the disclosed heat and compression therapy warp may be similarly integrated into a glove, elbow wrap, shoulder wrap, chest wrap, or any possible wraps or straps for providing heat and compression therapy for the corresponding parts. In addition, the heat and compression therapy wrap disclosed herein for the sandal, glove, or other wraps may or may not include cooling elements. For example, these warps may include only heat trace for heating therapy combined with compression therapy without necessarily providing cooling therapy. In some embodiments, even compression therapy may not be included in the wraps disclosed herein. The various configurations for these heat and compression therapy wraps are not limited in the disclosure.

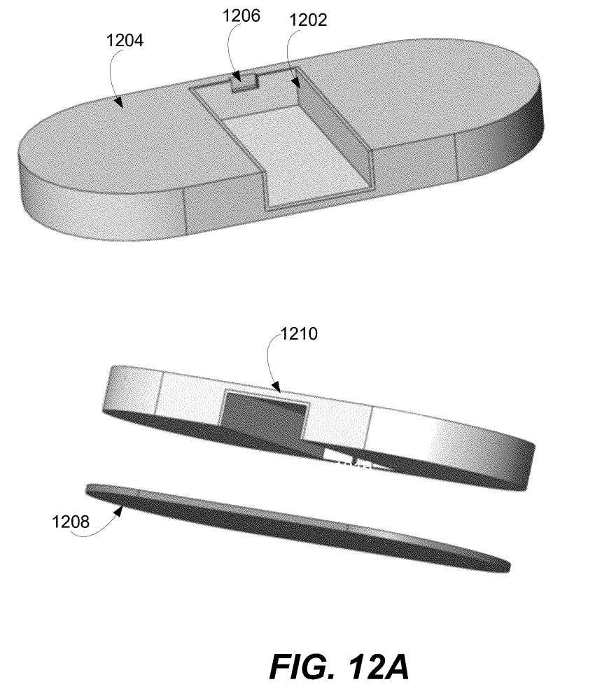

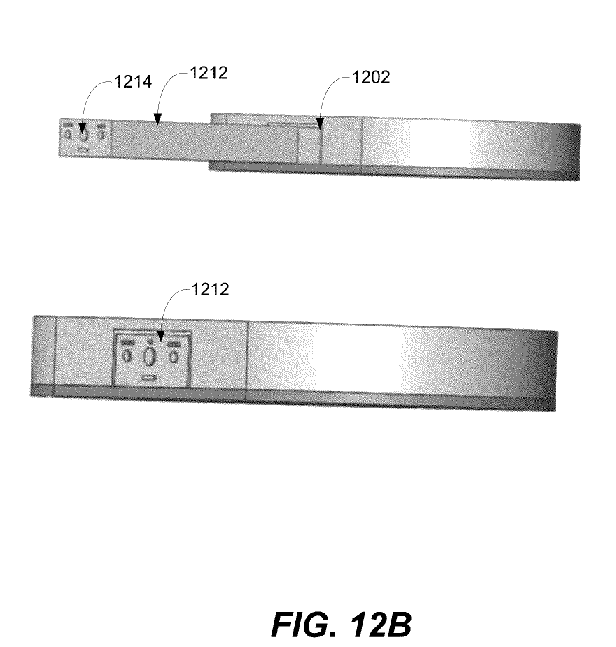

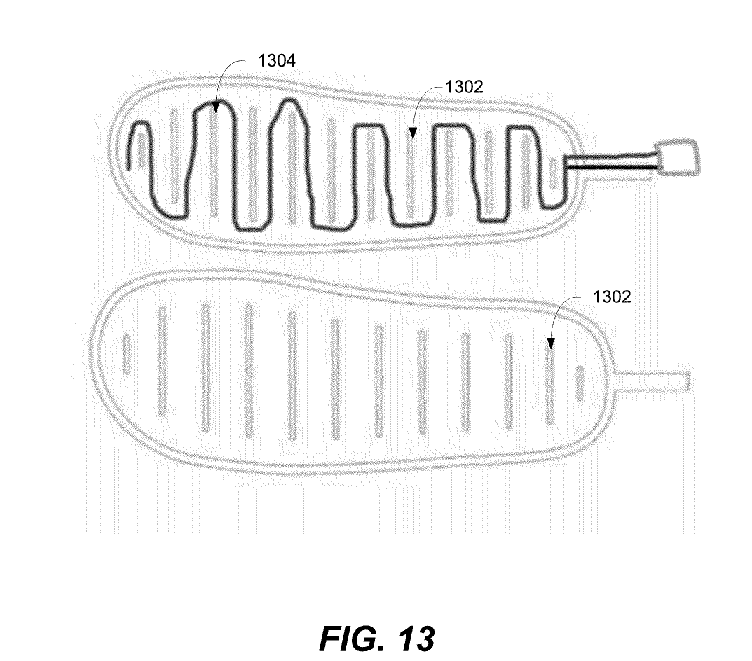

In the following, various configurations for integrating electronics into footwear are further described in FIGS. 12A-13. Specifically, FIGS. 12A-12B illustrate an example plastic dock for holding electronic and pneumatic connectors in a midsole. As can be seen from the top part in FIG. 12A, a rigid plastic dock 1202 may be glued inside a pre-casted cavity in a shoe's midsole 1204. The dock may additionally include one or more locking features 1206, which may be a click locking feature or other types of locking features. When an outsole 1208 is placed over the rigid plastic dock 1202, the dock becomes less obvious/visible from the outside, ensuring a sleek look. While not shown in FIG. 12A, according to some embodiments, the rigid plastic dock 1202 may further include one or more connection ports disposed along the sidewalls of the plastic dock 1202, where these ports are configured for connection with the electronic and/or pneumatic connectors once these connectors are placed inside the plastic dock 1202.

According to some embodiments, by using a rigid plastic dock, it may ensure the held electronic and pneumatic parts are not easily pressed by a person who wears the shoe, thereby improving the lifecycle of the components included therein. In some embodiments, the part 1210 of the midsole over the plastic dock may be configured to have a thickness that is sufficient so that a person who wears the shoe does not feel uncomfortable due to the harder part of the rigid plastic dock 1202 thereunder. In some embodiments, the part 1210 of the midsole over the plastic dock and its immediate surroundings may be configured to have an elasticity higher than the remaining part of the midsole, so that the midsole is not easy to break from the thinner part 1210 of the midsole.

It should be noted that while the rigid plastic dock 1202 is illustrated to have a box shape, the disclosure is not limited to such a shape. Instead, the plastic dock 1202 disclosed herein may have any possible shape, either regular or irregular, as long as the dock can hold the electronic and pneumatic parts as compact as possible and as tight as possible. In addition, while the plastic dock 1202 is shown to be disposed in the middle part of the midsole, in real applications, the plastic dock 1202 may be placed anywhere in the midsole, depending on the design needs. According to some embodiments, the plastic dock 1202 may be placed in a part corresponding to the arch of a foot so that less pressure may be placed on the plastic dock 1202 from a person who wears the shoe.

FIG. 12B illustrates an exterior battery pack 1212 to be placed inside the plastic dock 1202, according to some embodiments. Depending on the configuration, the exterior battery pack 1212 may hold one or more of a battery, a vibration sensor, one or more control buttons 1214, a compressor, one or more solenoids, a pressure sensor, an accelerometer, etc. The various components together may provide a precise control over the pressure delivered to a compression therapy wrap as discussed in FIGS. 11A-11B. According to some embodiments, a wireless communication unit may be further included to allow fine tuning or control of the components included therein. In addition, the battery included therein may be configured to allow a wireless charge to minimize the wires and/or connection ports included in the battery pack 1212 and plastic dock 1202.

According to some embodiments, the exterior battery pack 1212 is configured to easily slide and lock into the plastic dock 1202. According to some embodiments, by keeping the electronics and shoe manufacturing separate, it allows a modular customization of the electronics. For example, if one or more parts included in the exterior battery pack 1212 are broken, the exterior battery pack 1212 may be easily pulled out to repair the broken part, or a whole new battery pack 1212 may replace the old one instead if the broken part is not easy to get fixed. In addition, based on the condition/preference changes for a person, different types of battery packs 1212 may be placed into the plastic dock 1202 at different time points for the same person. The different types of battery packs may include different functions provided through the battery pack. For example, one battery pack 1212 may include a vibrator while another battery pack may not have a vibrator, both of which may be slid into the same lock, which allows a user to choose different battery packs at different time points.

It should be noted, while the slot for sliding the battery pack 1212 is shown to be located on the side of the shoe, the disclosure is not limited to such a configuration. In real applications, the slot can be located at any possible location, depending on where the plastic dock 1202 is disposed.

It should be also noted, while the plastic dock 1202 and the exterior battery dock 1212 are described with reference to the slider/sandal/flip-flop shown in FIGS. 11A-11B, such configuration is limited to slider/sandal/flip-flop applications, but can be applied to any type of shoes. For example, the plastic dock 1202 and exterior battery pack 1212 disclosed herein may be used to replace the electronic control unit in the heel clip as described in FIGS. 9A-9D.

Referring now to FIG. 13, an example heater bladder footbed is further described. As shown in the figure, the shoe in FIG. 13 integrates an inflatable air bladder 1302 with an embedded heating element 1304 to enhance warmth and comfort. This design ensures better heat distribution, customized foot support, and efficient energy use.

According to some embodiments, the heated bladder footbed may include an outer layer (or top layer) that is made of memory foam, synthetic mesh or leather for comfort, which also allows heat transfer from the heating element 1304 to the foot. Under the outer layer, there is an inflatable air bladder (middle layer) which includes a thin, flexible air chamber inside the midsole, which can be inflated using a mini air pump or pressor regulator. When inflated, the inflatable air bladder may push the heating element closer to the foot for better warmth. According to some embodiments, the pressure inside the bladder may be customized for arch support or shock absorption.

For the specific heating element 1304, it may be a flexible heating wire that is placed away from the ladder welding seams to avoid damage to the heating element. The heating element 1304 may be controlled to generate gentle, controlled heat, e.g., regulated by temperature sensors. The materials used to make the heating element 1304 may include certain carbon fibers or graphene-based films or the like that can reduce power consumption of the heating element 1304.

For the temperature sensor and control unit, they may be built-in units that monitor the foot temperature. According to some embodiments, the control unit may be configured to auto-adjust the heating level of the heating element 1304 to prevent overheating. Additionally, the control unit may also be configured to allow a user to control (e.g., adjust a desired temperature or temperature range) via buttons on the shoe or through a mobile app. Under certain circumstances, an AI-powered unit may be included in the control unit to provide a more customized control based on the user preference.

For the power source, it may be powered by a rechargeable battery, which may be integrated, or may be external as described earlier in FIGS. 12A-12B. In addition, wireless charging may be configured to eliminate external ports, making the shoe sleeker.

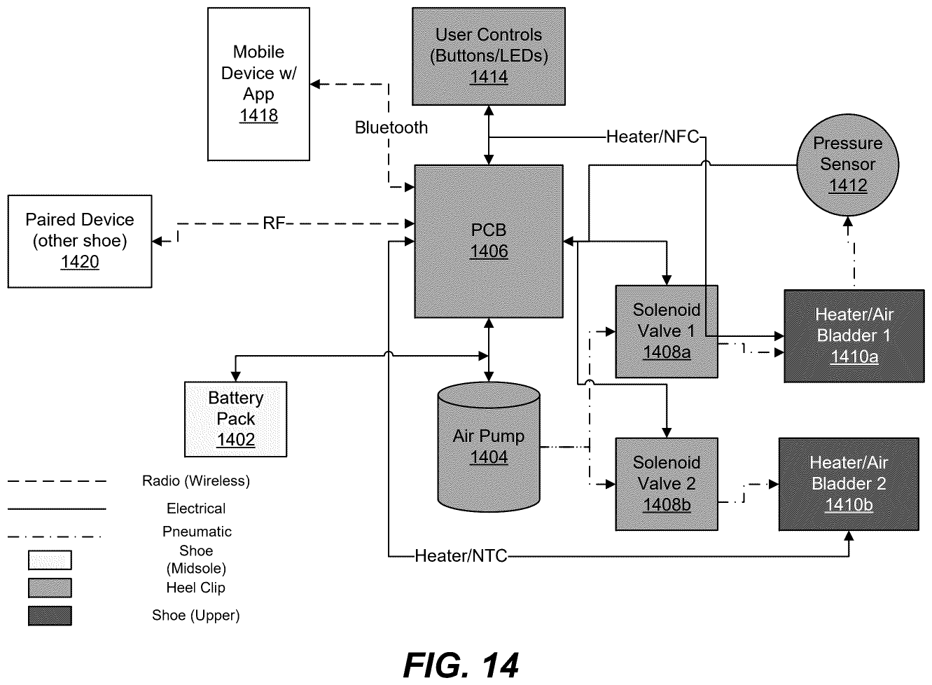

Referring now to FIG. 14, a block diagram of an exemplary heat and compression therapy apparatus 100 is illustrated. The exemplary heat and compression therapy illustrated in the figure may correspond to a heat and compression therapy apparatus shown in FIGS. 7A-13. As illustrated in the figure, the apparatus 100 includes a battery pack 1402, which may correspond to the battery pack 1008 shown in FIG. 10A. The battery pack 1402 provides power to different components in the apparatus 100. For example, as illustrated, the battery pack 1402 provides power to air pump 1404 (also referred to as “compressor” throughout the specification), which can inflate a bladder (e.g., a specific zone shown in FIGS. 7A-8C). The battery pack 1402 also provides power to a printed circuit board (PCB) 1406, which may be considered the central signal processing and control unit for the disclosed heat and compression therapy apparatus 100. For example, user inputs received through the user controls such as the buttons 908 are processed in the PCB, which further controls the operation of the air pump 1404 and heater/air bladder 1410a or 1410b. More specifically, the PCB 1406 generates control signals to control the power applied to the air pump 1404 and the power provided to the heat generation unit such as heater/air bladder 1 1410a and heater/air bladder 2 1410b. In some embodiments, the PCB 1406 also receives the pressure information sensed by the pressure sensor 1412. The sensed information allows to further control the air pump 1404 by the PCB 1406.

In some embodiments, the air pump 1404 inflates the air bladder 1 1410a through a solenoid valve 1 1408a and inflates the air bladder 2 1410b through a solenoid valve 1408b. The solenoid valve 1 can be energized to open the airway from the compressor to air bladder 1 1410a and close the path to the atmosphere in order to pressurize air bladder 1 1410a. Similarly, solenoid valve 2 can be energized to open the airway from the compressor to air bladder 2 1410b and close the path to the atmosphere in order to pressurize air bladder 1410b. In some embodiments, the PCB 1406 also provides control signals to de-energize solenoid valve 1 or solenoid valve 2 to release air from air bladder 1410a or 1410b.

In some embodiments, the PCB 1406 also communicates with external components through wireless communications. For example, as illustrated in FIG. 14, the PCB 1406 can communicate with a mobile device (which is installed with a proper app for controlling the apparatus 100) 1418 through Bluetooth, ANT+, or other different means. ANT+ is a wireless technology that allows monitoring devices to talk to each other. ANT+ stands for interoperability which means that ANT+ products from multiple brands work together. Leading brands design ANT+ into top products to ensure timely data delivery.

In some embodiments, the PCB 1406 can also communicate with another paired/coupled apparatus 1420 (e.g., another shoe, jacket, jogger, or a vest with a heat and compression therapy function, or another wearable device with or without a heat and compression therapy function) through a radio frequency (RF) signal, ANT+, or other different means or through a synchronization tool. HyperSync® is one of the technologies that allow for hyper-speed syncing. More specifically, through wireless communication or different synchronization tools, the pair/couple can talk to each other so that when a person is under treatment. For example, the respective controllers can communicate with each other in real time to make sure that the two in the pair are working within around a same timeline and/or even following a similar tempo or pattern (that is, the two are “synchronized”). It should be noted that, under certain circumstances, the two in the air can still operate independently. For example, if just one foot gets hurt, a user may expect treatment for one foot only. At this point, the two shoes in the pair may be not “synchronized”.

As also illustrated in FIG. 14, the heater/air bladder 1/heater/air bladder 2 are disposed in the shoe upper, the user controls 1414, the PCB 1406, the air pump 1404, the solenoid valve 1/solenoid valve 2, and pressure sensor 1412 are disposed in the heel clip, and the battery 1402 is disposed in the midsole of a shoe.

Referring now to FIG. 15, the control buttons and LEDs 1500 for controlling the heat and compression therapy apparatus 100 are further described. As illustrated in the figure, the control buttons include a power button 1502, a compression level button 1506, a heat level button 1510, and a start/stop button 1514. The power button 1502 also has an associated LED indicator 1504 to indicate the battery status level. The compression level button 1506 also has an associated set of LED indicators 1508 to indicate different predefined compression levels that can be applied to the human body. The heat level button 1510 also has a set of associated LED indicators 1512 to indicate different predefined heat levels that can be applied to the human body. The start/stop button 1514 also has a corresponding LED indicator 1516 to indicate the start/stop state of the heat and compression therapy apparatus 100 while the apparatus is powered on. It should be noted that the control buttons and LEDs shown in FIG. 15 are merely for exemplary purposes but not for limitations. For example, in some embodiments, there may be an additional redlight indicator to indicate possible errors or problems that occurred to the disclosed heat and compression therapy apparatus. In addition, if there are additional functions integrated into or combined with the disclosed heat and compression therapy apparatus, there may be additional control buttons or LEDs associated with these functions. For example, there may be a steam button to control steam (or heated water)-based thermal therapy if it is included in the disclosed heat and compression therapy apparatus.

In some embodiments, to turn on the heat and compression therapy apparatus 100 when the apparatus is in the off state, a user can press and hold the power button 1502 for a certain period of time (e.g., 500 ms or another preset time period) or longer (but less than a period for system reset). The LED 1504 can exhibit a color (e.g., a blue or green or another different color) to indicate that the apparatus is turned on. In addition, the LED 1504 can also indicate the battery level, for example, through a percentage of the filled color in the LED indicator frame and/or through different colors (e.g., a yellow color may indicate the battery is between 25-75% in capacity, an orange color may indicate that the battery is at 25% capacity or less, etc.). To turn off the heat and compression therapy apparatus 100, a user can similarly press and hold the power button 1502 for a certain period of time (e.g., 500 ms or another preset time period) or longer (but less than a period for system reset). After the apparatus 100 is turned off, the power level indicator 1504 is also off, and does not show any color. However, under certain circumstances, the battery for the apparatus may be in charge while the heat and compression therapy apparatus 100 is turned off. At this moment, the power level indicator 1504 still keeps on indicating the battery charging level while the apparatus is turned off.

In some embodiments, the power button 1502 can also function as a reset button to hard reset the heat and compression therapy apparatus 100. For example, under certain circumstances, there may be certain fault conditions that can be resolved through system resetting. At this moment, a user can push and hold the power button 1502 for a relatively longer time (e.g., 10 seconds or another predefined time period) or a system reset. This then forces the apparatus 100 to quit the operation and reboot the microcontroller unit included in the apparatus to recover the apparatus 100.

Here the following are example settings for the power level indicator 1504. It should be noted that the present disclosure is not limited to such settings for the power level indicator 1504.

Power/Battery LED

| | | On/Running (Device | Charging (Device is |
|---|---|---|---|
| | Battery | is ON, in Idle, Pause | OFF, Plugged in to |
| | Level | or Running) | Charger) |
| Status | Low Battery | Orange* (solid) | Orange (breathing) |
| | 25%-75% | Yellow (solid) | Yellow (breathing) |
| | 75%-99% | Green (solid) | Green (breathing) |
| | 100% | Green (Solid) | Green (Solid) |

*When Low Battery occurs, if a session is running, the device will stop the session, and shut off the heaters, pump, and valves. Will disable all buttons aside from the Power button.

In some embodiments, a user can also control the compression level provided by the heat and compression therapy apparatus 100 to a user through the compression level button 1506. For example, a user can short-press the compression level button once to allow the apparatus to provide a first-level compression. At this moment, the first LED of the set of LEDs 1508 can exhibit a color (which can be a color different from the power level indictor, such as a white color or another different color) to indicate that the first level of compression is to be provided to the user. At this point, if the user short presses the compression level button 1506 again, the compression level can be switched to a second level. At this moment, a second LED of the set of LEDs 1508 can exhibit a color (at this moment, the first LED may or may not exhibit a color). To continue to switch to another level, the user can further short-press the compression level button 1506 again, which allows a third-level compression to be provided to the user. At this moment, a third LED of the set of LEDs 1508 can exhibit a color (the first and second LEDs may or may not exhibit a color). If the user continues to short-press the compression level button 1506, the heat and compression therapy apparatus 100 can enter the “walking mode,” which is a mode that allows a user to walk with the shoe without necessarily going through the treatment by the apparatus 100 included in the shoe. In this mode, no compression is provided to the user, and no LED 1508 exhibits a color or is lighted up. In summary, the compression level button 1506 may be a cyclical loop button that switches between “-Off-1-2-3-Off” levels or states, where the off state may correspond to the walking mode of the heat and compression therapy apparatus 100.

It is to be noted that, while three different levels of compression are described here, in some embodiments, there can be more or less than three levels of compression that can be provided by the heat and compression therapy apparatus 100. In some embodiments, there can be no predefined set of compression levels as described above. Instead, the compression level button 1506 may include a pair of buttons, where pressing the first button of the pair of buttons can continuously increase the compression level to be provided to a user while pressing the second button can continuously decrease the compression level to be provided to the user. In such circumstances, the compression level indicator 1508 may not be a series of LED indicators as shown in FIG. 15, but can be a single LED similar to the power level indicator 1504. That is, the exact level of compression to be provided by the heat and compression therapy apparatus 100 can fall between any desired level between the maximum compression level and zero compression level.

Here the following are example settings for the compression level indicator 1508. It should be noted that the present disclosure is not limited to such settings for the compression level indicator 1508.

| | |
|---|---|
| | Target Pulse/Inflate |
| Pressure | Pressure (mm Hg) |

| Level | Zone 1 | Zone 1, 2 |
|---|---|---|
| 1 | 60 | 50 |
| 2 | 130 | 120 |
| 3 | 200 | 190 |

It is to be noted that, the levels in the table are for “compression massage mode” (when both heat and compression are on). When the heat and compression therapy apparatus 100 is in the “compliance mode” (when the heat is on, but the compression is off), the bladders in both zones are inflated to 30 mm HG and held. Under certain circumstances, when the heat and compression therapy apparatus 100 operates under the walking mode, the pressure can be set to 30 mm HG, and when the heat and compression therapy apparatus 100 operates under the heat-only mode, the pressure can be set to 40 mm HG.

In some embodiments, a user can also control the heat level provided by the heat and compression therapy apparatus 100 to a user through the heat level button 1510. For example, a user can short-press the heat level button 1510 once to allow the apparatus to provide first-level heat. At this moment, the first LED of the set of LEDs 1512 may exhibit a color (which can be a color different from the power level indictor, such as a white color or another different color), to indicate that the first level of heat is to be provided to the user. At this point, if the user short presses the heat level button 1510 again, the heat level to be provided to the user can switch to a second level. At this moment, a second LED of the set of LEDs 1512 may exhibit a color (at this moment, the first LED may or may not exhibit a color). To continue to switch to another level, the user can further short-press the heat level button 1510, which allows a third-level heat to be provided to the user. At this moment, the third LED of the set of LEDs 1512 may exhibit a color (the first and second LEDs may or may not exhibit a color). If the user continues to short-press the heat level button 1510, the heat and compression therapy apparatus 100 can enter into an off state, which is a state where no heat is provided to the user. At this moment, no LED 1512 exhibits a color or is lighted up. In summary, the heat level button 1510 may be a cyclical loop button that switches between “-Off-1-2-3-Off” levels or states.

It is to be noted that, while three different levels of heat are described here, in some embodiments, there can be more or less than three levels of heat that can be provided by the heat and compression therapy apparatus 100. In addition, in some embodiments, there can be no predefined different heat levels. Instead, the heat level button 1510 may include a pair of buttons, where pressing the first button can continuously increase the heat level to be provided to a user while pressing the second button can continuously decrease the heat level to be provided to the user. Under such circumstances, the heat level indicator 1512 may not be a series of LED indictors as shown in FIG. 15, but can be a single LED similar to the power level indicator 1504. That is, the exact level of heat to be provided by the heat and compression therapy apparatus 100 can fall between any level between the maximum heat level and zero heat level.

Here the following are example settings for the heat level indicator 1512. It should be noted that the present disclosure is not limited to such settings for the heat level indicator 1512.

| | |
|---|---|
| | Target |
| Heat | Temperature (f.) |

| Level | Zone 1 | Zone 2 |
|---|---|---|
| 1 | 104 | 104 |
| 2 | 113 | 113 |
| 3 | 122 | 122 |

It is also to be noted that while the temperatures are shown to be the same for two zones, under certain circumstances, the two zones can be configured to have different temperatures so that each zone can be independently controlled, as described earlier.

In some embodiments, instead of setting target temperatures and measuring the temperatures in different zones (or different areas for certain wear), it is also possible to measure trends in amperage to maintain temperature in these zones or areas. The underlying theory of amperage measurement is that it will take more energy to maintain temperature if your tissue is colder.

In some embodiments, the heat and compression therapy apparatus 100 further includes a start/stop button 1514 and a corresponding LED indicator 1516. For example, after the compression level and the heat level are adjusted to a desired level, a user can then press the start/stop button 1514 to start a session of treatment, which lasts a predefined period of time (e.g., 15 minutes or another different period). After the current session is done, the heat and compression therapy apparatus 100 can enter into an idle state or stand-by mode before entering into the next session of treatment. In some embodiments, a user may desire to adjust the compression level and/or heal level for the next session. In some embodiments, a user can pause an ongoing session by pushing the start/stop button 1514, which can either cancel the current session or pause the ongoing session (e.g., depending on how long the start/stop button 1514 is pressed). In some embodiments, depending on the configuration, there can be no pause option and the heart and compression therapy apparatus 100 directly cancels the ongoing session when the start/stop button 1514 is pressed by the user while there is still an ongoing session.

In some embodiments, when a user presses the start/stop button 1514, the expected session may not proceed as expected. Instead, there is a possible error, which then causes one or more LED indicators to blink. Possible error can also happen when an ongoing process is in process, which then causes the heat and compression therapy apparatus to stop the session, and shut off the heaters, pumps, and/or valves for safety concerns.

Here the following are some exemplary errors that may occur to the disclosed heat and compression therapy apparatus 100. In one example, an air leak may occur in a first zone, and thus the pressure sensor does not receive the target pressure in the time period allowed. At this moment, the apparatus stops operating and the compression level indictor LED 1 may flash, to indicate that there is an air leak in the first zone. In another example, an air leak may occur in a second zone. At this moment, the apparatus stops operating and the compression indictor LED 2 may flash, to indicate that there is an air leak in the second zone. Under certain circumstances, a pressure sensor tube may be blocked, which can cause no reading (or every low reading value) from the pressure sensor. At this point, the first compression level indicator LED1 becomes flashing, indicating the possible error.

In yet another example, the temperature sensor may not work, which can be identified when the first heat level indicator LED1 flashes. For example, the temperate sensor or wires connected to the sensor may be damaged, dislodged, or become defective, which then causes the NTC not to read. The first heat level indicator LED1 then turns to flash, indicating that the temperature sensor is not working. In yet another example, the heater itself may not work, which can cause the NTC to read the room temperature. To report this problem, the second heat level indicator LED2 can be caused to flash. In yet another example, the temperature may be out of bounds, which may cause the NTC to read a wired value. This problem can be identified when the third heat level indicator LED3 flashes. In some embodiments, there may be other problems, not listed above, that can be found in the heat and compression therapy apparatus 100, which can be identified through the LED blink of one LED or a combination of two or more LEDs if necessary.

In some embodiments, after a user selects desired heat and/or compression levels, the user may push the start/stop button 1514 to start a treatment session. In some embodiments, a session of treatment performed by the heat and compression therapy apparatus 100 may be controlled through a set of predefined events to control the pressure applied to the air bladders in the apparatus 100. In one example, a “Massage Treatment Algorithm” may be predefined, which allows a series of events to be performed by the heat and compression therapy apparatus during the treatment. The apparatus 100 may thus be controlled to perform these events sequentially to complete a treatment session following the massage treatment algorithm. In the following, a number of exemplary events are specifically described. However, it is to be noted that the heat and compression therapy apparatus 100 is not limited to these events during a treatment session, but can actually include fewer events or more events not described below. In addition, these events are configured with reference to the heat and compression therapy apparatus shown in FIGS. 7A-13. If another different heat and compression therapy apparatus is configured. There can be different events than those described below. Further, certain values and/or numbers in the described events are for exemplary purposes, and not for limitation.

With respect to Event 0, the heat and compression therapy apparatus 100 (also referred to as “device” throughout the specification) is in the off/idle state, and the event is also referred to as “Off/Idle” event. During this event:
Device is either OFF or Idle.
All valves and the compressor are OFF
The normal state of valves when OFF (not energized) is to open the airpath between air bladders in the wrap to the atmosphere
   This allows the air bladders to de-pressurize whenever treatment is stopped or the device is Off.
Pressing the start button causes the device to move to the next event (i.e., Event 1)
All valves and the compressor are OFF
Normal state of valves when OFF (not energized) is to open the airpath between air bladders in the wrap to the atmosphere.
Pressing the start button causes the device to move to the next event (i.e., Event 1)
   During this event, the compressor is off, and Valve 1 and Valve 2 are not energized. To move to the next event, the start/stop button can be pressed. In some embodiments, once one unit (i.e., apparatus 100 in one shoe) is started, the other one will be started synchronously through RF communication.

With respect to Event 1, Valve 1 included in the apparatus 100 is energized to inflate air to Zone 1, and the event is also referred to as “V1 Pulse Inflate” event. During this event:
Compressor is ON
Valve 1 is energized, opening the airway from the compressor to Zone 1 of the air bladder and closing the path to the atmosphere in order to pressurize Zone 1
Pressure Sensor monitors for target pressure
Once target pressure is achieved, move to the next event (i.e., Event 2)
   During this event, the compressor is off, Valve 1 is energized, and Valve 2 is not energized. To move to the next event, pressure is continuously provided by the compressor.

With respect to Event 2, the pressure in Zone 1 is held, and the event is also referred to as “V1 Pulse Hold” event. During this event:
Compressor is OFF
Valve 1 is energized closing the path to the atmosphere so that the air bladder does not lose pressure
Start internal Time count.
Once Time is achieved, move to the next event (i.e., Event 3)
   During this event, the compressor is off, Valve 1 is energized, and Valve 2 is not energized. Before moving to the next event, there may be a “Time 1” delay, which can last 2 seconds or another predefined value. In some embodiments, when both units finish the Hold process, both enter the next event at the same time by RF communication, that is, both units do not enter the next event (Release) until both finish the Hold process.

With respect to Event 3, the pressure in Zone 1 is released, and the event is also referred to as “V1 Pulse Release” event. During this event:
Compressor is OFF
Valve 1 is de-energized opening the path to the atmosphere in order to de-pressurize Zone 1
Start internal Time count.
Once Time is achieved, move to the next event (Event 4)
   During this event, the compressor is off, and both Valve 1 and Valve 2 are not energized. Before moving to the next event, there may be a “Time 2” delay, which can last 4.5 seconds or another predefined value. In some embodiments, when both units finish the Release process, both enter the next event at the same time by RF communication, that is, both units do not enter the next event until both finish the Release process.

With respect to Event 4, the events including Event 1-Event 3 are repeated multiple times, and the event is also referred to as “Loop V1 Pulse Events X 1” event. During this event:
Events 1-3 create the “Pulsing” sensation of compression in Zone 1
Once Events 1-3 have occurred 3 times (or whatever repetitions the user sets), move to the next event (Event 5)

With respect to Event 5, Valve 1 included in the apparatus 100 is further energized to inflate air to Zone 1 to increase 10 mm Hg more of the pressure, and the event is also referred to as “V1 Hold Inflate” event. During this event:
Compressor is ON
Valve 1 is energized opening the airway from the compressor to Zone 1 of the air bladder and closing the path to the atmosphere in order to pressurize Zone 1
Pressure Sensor monitors for target pressure
Target Pressure is equal to the Pulse Inflate Pressure (Event Number 1)+10 mm Hg e.g., if Pulse Inflate target pressure is 80 mm Hg, then Hold Inflate target pressure is 90 mm Hg
Once target pressure is achieved, move to the next event
Once target pressure is achieved, move to the next event (i.e., Event 6)
   During this event, the compressor is on, Valve 1 is energized, and Valve 2 is not energized. Before moving to the next event, pressure is continuously provided by the compressor.

With respect to Event 6, the pressure in Zone 1 is held, and the event is also referred to as “V1 Hold” event. During this event:
Compressor is OFF
Valve 1 is energized, closing the path to the atmosphere so that the air bladder does not lose pressure
Start internal Time count.
Once Time is achieved, move to the next event (i.e., Event 7)
   During this event, the compressor is off, Valve 1 is energized, and Valve 2 is not energized. Before moving to the next event, there is a “Time 3” delay, which can last 4 seconds or another predefined time. In some embodiments, when both units finish the Hold process, both enter the next step at the same time by RF communication, that is, both units do not enter the next step until both finish the Hold process.

With respect to Event 7, both Zone 1 and Zone 2 are pressured, and the event is also referred to as “V1; V2 Pulse Inflate” event. During this event,
Compressor is ON
Valves 1 and 2 are energized, opening the airway from the compressor to Zone 1 and Zone 2 of the air bladder and closing the path to the atmosphere in order to pressurize Zones 1 and 2
Some of the air in bladder 1 will very quickly enter bladder 2 causing a reduction in pressure Valve 1, but giving bladder 2 a “head start” in inflating beyond what the compressor alone would achieve
The pressure in bladder 1 will be greater than bladder 2 creating a gradient pressure from distal to proximal until the pressures in both bladders equalize.
Once both bladders have achieved equal pressure, they will stay equal while both bladders fill until target pressure is achieved and remain equal for the duration of the Pulse Inflate
Pressure Sensor monitors for target pressure.
Once target pressure is achieved, move to the next event (i.e., Event 8)
   During this event, the compressor is on, and Valve 1 and Valve 2 are both energized. Before moving to the next event, the pressor is continuously provided by the compressor.

With respect to Event 8, pressure is held for both Zone 1 and Zone 2, and the event is also referred to as “V1; V2 Pulse Hold” event. During this event:
Compressor is OFF
Valve 1 and 2 are energized opening the airway from the compressor to Zone 1 and Zone 2 of the air bladder and closing the path to the atmosphere in order to pressurize Zones 1 and 2
   Some of the air in bladder 1 will very quickly enter bladder 2 causing a reduction in pressure Valve 1, but giving bladder 2 a “head start” in inflating beyond what the compressor alone would achieve
   The pressure in bladder 1 will be greater than bladder 2 creating a gradient pressure from proximal to distal until the pressures in both bladders equalize
   Once both bladders have achieved equal pressure, they will stay equal while both bladders fill until target pressure is achieved
Pressure Sensor monitors for target pressure.
Once target pressure is achieved, move to the next event (i.e., Event 9)
   During this event, the compressor is off, and both Valve 1 and Valve 2 are energized. Before moving to the next event, there is a “Time 1” delay, which can last 2 seconds or another predefined time. In some embodiments, when both units finish the Hold process, both enter the next step at the same time by RF communication, that is, both units do not enter the next step (Release) until both finish the Hold process.

With respect to Event 9, the pressure in both Zone 1 and Zone 2 is released, and the event is also referred to as “V1; V2 Pulse Release” event. During this event:
Compressor is OFF
Valves 1 and 2 are de-energized opening the path to the atmosphere, and de-pressurizing Zones 1 and 2
Start internal Time count.
Once Time is achieved, move to the next event
   During this event, the compressor is off, and Valve 1 and Valve 2 are not energized. Before moving to the next event, there is a “Time 2” delay, which can last 4.5 seconds or another predefined time. In some embodiments, when both units finish the Release process, both enter the next step at the same time by RF communication, that is, both units do not enter the next step until both finish the Release process.

With respect to Event 10, the events including Event 7-Event 9 are repeated multiple times, and the event is also referred to as “Loop V1; V2 Pulse Events X 2” event. During this event:
Events 7-9 create the “Pulsing” sensation of compression in Zone 1 and Zone 2.
Once Events 7-9 have occurred 3 times (or whatever repetitions the user sets), move to the next event (Event 11)

With respect to Event 11, Valve 1 and Valve 2 included in the apparatus 100 are further energized to inflate air to Zone 1 to 10 mm Hg more, and the event is also referred to as “V1; V2 Hold Inflate” event. During this event:
Compressor is ON
Valves 1 and 2 are energized opening the airway from the compressor to zone 1 and zone 2 of the air bladder and closing the path to the atmosphere in order to pressurize zones 1 and 2
Pressure Sensor monitors for target pressure
Target Pressure is equal to the Pulse Inflate Pressure (Event Number 7)+10 mm Hg e.g. if Pulse Inflate target pressure is 80 mm Hg, then Hold Inflate target pressure is 90 mm Hg
Once target pressure is achieved, move to the next event (i.e., Event 12)
   During this event, the compressor is on, and Valve 1 and Valve 2 are energized. Before moving to the next event, pressure is continuously provided by the compressor.

With respect to Event 12, pressure in Zone 1 and Zone 2 is held, and the event is also referred to as “V1; V2 Hold” event. During this event:
Compressor is OFF
Valves 1 and 2 are energized closing the path to the atmosphere so that air bladders do not lose pressure
Start internal Time count.
Once Time is achieved, move to the next event (i.e., Event 19)
   During this event, the compressor is off, and Valve 1 and Valve 2 are energized. Before moving to the next event, there is a “Time 3” delay, which can last 4 seconds or another predefined time. In some embodiments, when both units finish the Hold process, both enter the next step at the same time by RF communication, that is, both units do not enter the next step until both finish the Hold process.

With respect to Event 19, a full treatment cycle is completed, and a new cycle is restarted if applicable, and the event is also referred to as “Rest” event. During this event:
Compressor is OFF
Valves 1, 2, and 3 are de-energized, opening the path from air bladders to the atmosphere in order to de-pressurize zones 1, 2 and 3
Start internal Time count.
Once Time is achieved, this is the end of one full Treatment Cycle; move back to Event 1 and repeat until the Treatment Session is complete as dictated by the Session Timer
   During this event, the compressor is off, and Valve 1 and Valve 2 are not energized. Before moving to the next event, there is a “Time 4” delay, which can last 12 seconds or another predefined time. In some embodiments, when both units finish the Rest process, both enter the next step at the same time by RF communication, that is, both units do not enter the next step until both finish the Rest process.

With respect to Event 20, the treatment is stopped, and the event is also referred to as “Stop treatment” event. During this event:
Device is Idle.

All valves and the compressor are OFF
Normal state of valves when OFF (not energized) is to open the airpath between air bladders in the wrap to the atmosphere
   This allows the air bladders to de-pressurize whenever treatment is stopped or the device is Off
Pressing the start button causes the device to move to the next event (i.e., Event 1)
All valves and the compressor are OFF
The normal state of valves when OFF (not energized) is to open the airpath between air bladders in the wrap to the atmosphere.
Pressing the start button causes the device to move to the next event (Event 1)
   During this event, the compressor is off, and Valve 1 and Valve 2 are not energized. Before moving to the next event, the Start/Stop button can be pressed. In some embodiments, once one unit is stopped, the other one will be stopped synchronously through RF communication.

In some embodiments, in the events described above, there is a slight delay between the compressor on and the valve actuation, where the delay can be 500 ms or another possible value. In addition, in some of the events, the number of pulse event loops is configured to be variable for each zone to support further customization of the number of “pulses” per zone.

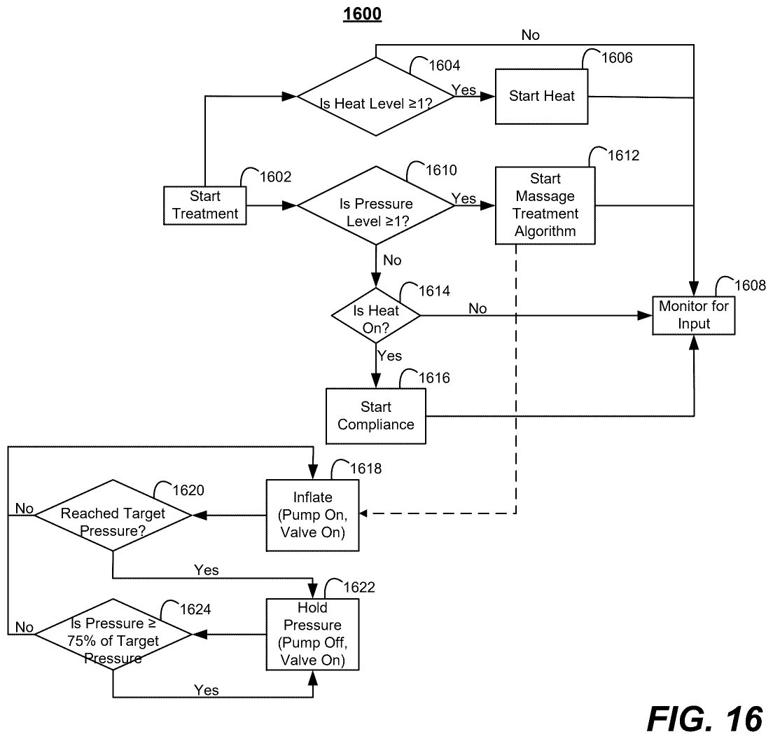

Referring now to FIG. 16, a flowchart of an exemplary method 1600 for performing treatment by the disclosed heat and compression therapy apparatus 100 is illustrated. The method 1600 starts at step 1602, where a user can push a start/stop power button 1502 to start the treatment process. Once the treatment process is started at step 1602, the apparatus 100 can first determine the target heat level and compression level set by the user. For example, the apparatus 100 may determine, at step 1604, whether the heat level set by the user is larger than or equal to the heat level 1. If it is determined that the set heat level is equal to or greater than the heat level 1, the apparatus 100 begins to generate and/or provide heat to the user at step 1606. If the heat level set by the user is not equal to or greater than 1, it means the user may have not set the heat level yet. At this moment, the heat and compression therapy apparatus 100 can monitor the user input at step 1608. For example, the user may find the heat level has not been set up, since the user does not feel any heat provided to the user after the user pushes the start/stop button at step 1602. Accordingly, the user may set up the desired heat level by pressing the heat level button 1506 one or more times. The apparatus 100 can start the heat generation after receiving the user input.

In some embodiments, the heat and compression therapy apparatus 100 can also check the pressure level set by the user at step 1610. If the apparatus 100 finds that the pressure level set by the user is equal to or greater than the pressure level 1, the apparatus 100 can start the compression treatment, e.g., following the massage treatment algorithm. If the pressure level is not equal to or greater than the pressure level 1, the apparatus 100 then checks whether the heater is turned on at step 1614. If the apparatus 100 is found to turn on the heat but no pressure level is provided, it may indicate that the user intends to use the compliance mode of the apparatus, and thus the apparatus 100 enters into the compliance mode at step 1616. However, if the apparatus finds that heat is not being generated, the apparatus may determine that the user has not provided proper levels for heat and compression, and thus continuously monitor the user input to wait for the desired heat and/pressure levels provided by the user at step 1608.

In some embodiments, when the apparatus 100 follows the massage treatment algorithm, the apparatus 100 may be required to provide a target level of pressure to a bladder or a specific zone of the bladder. Steps 1618-1624 describe an approach to providing the target pressure level to a bladder. The process may start at step 1618 to inflate a bladder or a specific zone of the bladder. For example, the compressor is turned on and the corresponding valve is energized. The pressure sensor begins to monitor the pressure level at the target zone or bladder, which allows to determine whether the target pressure level is reached or not at step 1620. If the target pressure level is reached, the apparatus 100 can hold the pressure at the target level at step 1622 by turning off the compressor while keeping the corresponding valve energized. Under certain circumstances, when the pressure is held inside a bladder or a zone, the pressure may decrease due to various reasons. Therefore, the pressure level is still monitored during the holding process. If it is found, at step 1624, that the pressure level falls below 75% of the target pressure level, the compressor can be turned on to inflate the bladder or the zone again to increase the pressure to the target level by returning to step 1618, as shown in FIG. 16.

It should be noted that, while the user can choose the desired levels of heat and pressure/compression for the treatment when defining different sets of heat levels and compression levels for user selection, it is possible for putting a preference for the energy to go toward temperature over pressure, since that is more efficient. For example, the set of heat levels defined for the apparatus may be relatively higher and can draw more energy when compared to the defined levels of compression or pressure. That is, in general, the temperature provided to a user is relatively high while the compression or pressure provided to the user is relatively low during treatment.

In addition, in some embodiments, the massage treatment algorithm disclosed herein may add the relationship to heat. That is, during the massage treatment sessions, the possible effect of the heat on massage outcome may be also considered when configuring the massage treatment algorithm, so as to further improve the performance of the disclosed heat and compression therapy apparatus 100.

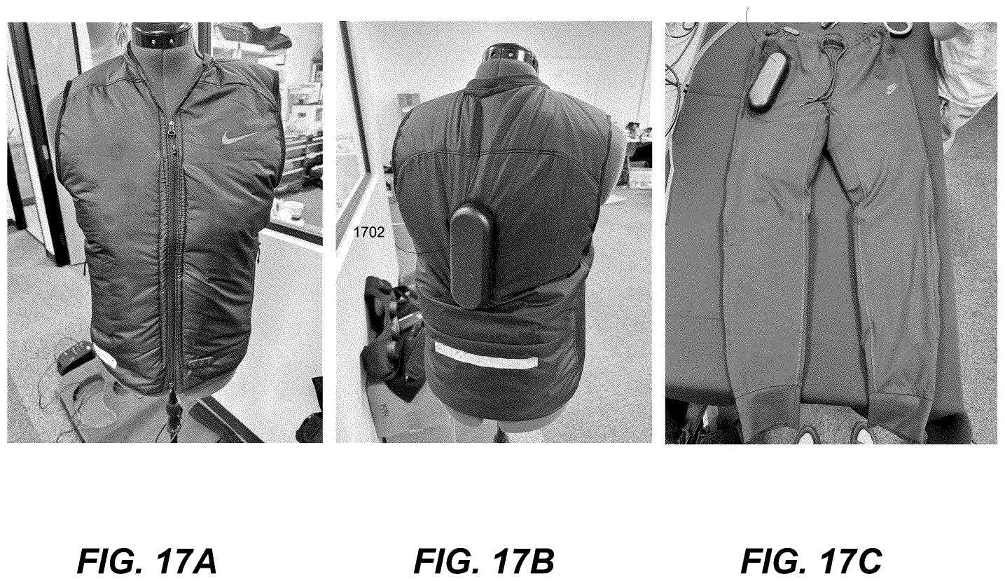

It should be also noted, while the heat and compression therapy apparatus 100 is mostly described with reference to a shoe for specific implementations, the present disclosure is not limited to such implementations. Instead, the heat and compression therapy apparatus 100 disclosed herein can be integrated into various sportwear, activewear, or other types of wear. FIGS. 17A-17B show prototype images of a vest with an integrated heat and compression therapy apparatus disclosed herein. As can be seen from FIG. 17B, the control unit, the battery, indicators, and certain other accessories are integrated into a lightweighted plastic box 1702, which is sewn onto the outer surface of the vest. The coupled bladders (not seen from the images) are sewn onto the inner surface of the vest with the heating pad facing the human skin. There can be one or more than one bladder sewn into different parts of the vest, for example, front and back parts around the waist area or chest area.

FIG. 17C shows a prototype image of a jogger with an integrated heat and compression therapy apparatus disclosed herein. As can be seen from the image, the plastic box 1702 is also sewn onto the outer surface of the jogger, with the bladder(s) sewn onto the inner surface of the jogger for heat and/or compression treatment purposes.

In some embodiments, besides the heat and compression therapy apparatus 100, other physical therapy and/or physiological monitoring apparatus may be integrated into or combined with the heat and compression therapy apparatus 100 disclosed herein. For example, certain sensors (e.g., electromyography (EMG) sensors or any other type of sensors) for measuring muscle strength or muscle output may be integrated into or combined with the heat and compression therapy apparatus 100 included in a jogger or vest or other type of wear. Additionally, or alternatively, certain nerve stimulation pads or needles may be also integrated into or combined with the heat and compression therapy apparatus 100 disclosed herein, so as to achieve additional benefits in promoting human health.

### Computer Systems

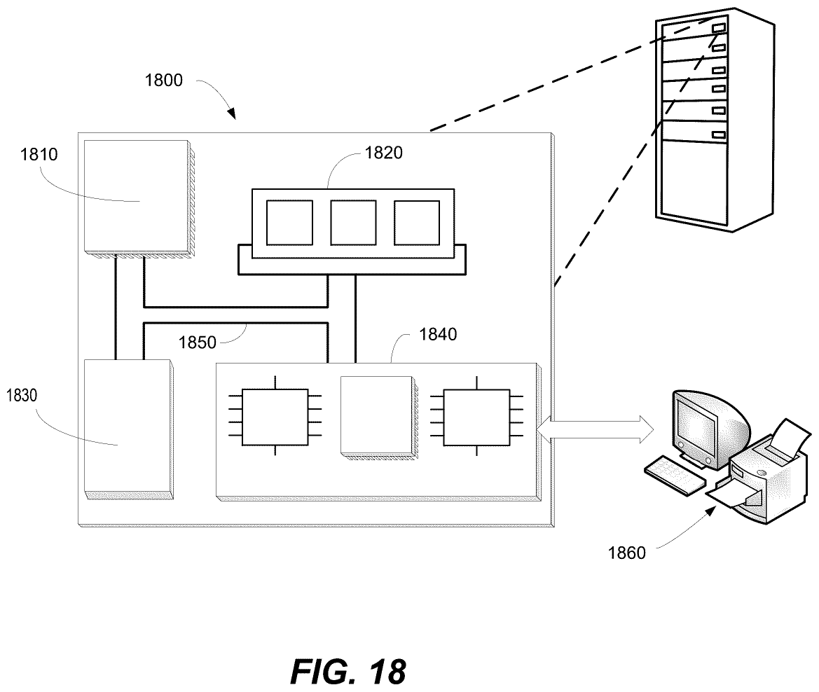

FIG. 18 is a block diagram of an example computer system 1800 that may be used in implementing the technology described in this document. General-purpose computers, network appliances, mobile devices, or other electronic systems may also include at least portions of the system 1800. The system 1800 includes a processor 1810, a memory 1820, a storage device 1830, and an input/output device 1840. Each of the components 1810, 1820, 1830, and 1840 may be interconnected, for example, using a system bus 1850. The processor 1810 is capable of processing instructions for execution within the system 1800. In some implementations, the processor 1810 is a single-threaded processor. In some implementations, the processor 1810 is a multi-threaded processor. The processor 1810 is capable of processing instructions stored in the memory 1820 or on the storage device 1830.

The memory 1820 stores information within the system 1800. In some implementations, the memory 1820 is a non-transitory computer-readable medium. In some implementations, the memory 1820 is a volatile memory unit. In some implementations, the memory 1820 is a nonvolatile memory unit.

The storage device 1830 is capable of providing mass storage for the system 1800. In some implementations, the storage device 1830 is a non-transitory computer-readable medium. In various different implementations, the storage device 1830 may include, for example, a hard disk device, an optical disk device, a solid-date drive, a flash drive, or some other large capacity storage device. For example, the storage device may store long-term data (e.g., database data, file system data, etc.). The input/output device 1840 provides input/output operations for the system 1800. In some implementations, the input/output device 1840 may include one or more network interface devices, e.g., an Ethernet card, a serial communication device, e.g., an RS-232 port, and/or a wireless interface device, e.g., an 802.11 card, a 3G wireless modem, or a 4G wireless modem. In some implementations, the input/output device may include driver devices configured to receive input data and send output data to other input/output devices, e.g., keyboard, printer and display devices 1860. In some examples, mobile computing devices, mobile communication devices, and other devices may be used.

In some implementations, at least a portion of the approaches described above may be realized by instructions that upon execution cause one or more processing devices to carry out the processes and functions described above. Such instructions may include, for example, interpreted instructions such as script instructions, or executable code, or other instructions stored in a non-transitory computer readable medium. The storage device 1830 may be implemented in a distributed way over a network, for example as a server farm or a set of widely distributed servers or may be implemented in a single computing device.

Although an example processing system has been described in FIG. 18, embodiments of the subject matter, functional operations and processes described in this specification can be implemented in other types of digital electronic circuitry, in tangibly embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible nonvolatile program carrier for execution by, or to control the operation of, data processing apparatus. Alternatively, or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “system” may encompass all kinds of apparatus, devices, and machines for processing data, including by way of example a programmable processor, a computer, or multiple processors or computers. A processing system may include special purpose logic circuitry, e.g., an FPGA (field programmable gate array) or an ASIC (application specific integrated circuit). A processing system may include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Computers suitable for the execution of a computer program can include, by way of example, general or special purpose microprocessors or both, or any other kind of central processing unit. Generally, a central processing unit will receive instructions and data from a read-only memory or a random-access memory or both. A computer generally includes a central processing unit for performing or executing instructions and one or more memory devices for storing instructions and data. Generally, a computer will also include or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship between client and server arises by virtue of computer programs running on the respective computers and having a client-server relationship to each other.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

### Terminology

The phraseology and terminology used herein is for the purpose of description and should not be regarded as limiting.

Measurements, sizes, amounts, and the like may be presented herein in a range format. The description in range format is provided merely for convenience and brevity and should not be construed as an inflexible limitation on the scope of the invention. Accordingly, the description of a range should be considered to have specifically disclosed all the possible subranges as well as individual numerical values within that range. For example, description of a range such as 1-20 meters should be considered to have specifically disclosed subranges such as 1 meter, 2 meters, 1-2 meters, less than 2 meters, 10-11 meters, 10-12 meters, 10-13 meters, 10-14 meters, 11-12 meters, 11-13 meters, etc.

Furthermore, connections between components or systems within the figures are not intended to be limited to direct connections. Rather, data or signals between these components may be modified, re-formatted, or otherwise changed by intermediary components. Also, additional or fewer connections may be used. The terms “coupled,” “connected,” or “communicatively coupled” shall be understood to include direct connections, indirect connections through one or more intermediary devices, wireless connections, and so forth.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “cither,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Having thus described several aspects of at least one embodiment of this invention, it is to be appreciated that various alterations, modifications, and improvements will readily occur to those skilled in the art. Such alterations, modifications, and improvements are intended to be part of this disclosure and are intended to be within the spirit and scope of the invention. Accordingly, the foregoing description and drawings are by way of example only.

What is claimed is:

1. A wearable thermotherapy apparatus, comprising:
at least one air bladder configured to conform to a contour of a portion of a user's body;
at least one thermal element disposed adjacent to the air bladder and configured to apply heat to a targeted portion of the user's body;
a support structure configured to limit outward expansion of the air bladder;
a control unit configured to regulate heat and compression delivered via the thermal element and air bladder;
a power source operably connected to the thermal element and control unit; and
a user interface comprising one or more buttons and indicators to control and display compression and heat levels.

2. The apparatus of claim 1, wherein the thermal element comprises resistance wires disposed between two fabric layers.

3. The apparatus of claim 1, wherein the thermal element is integrated into the air bladder through adhesive bonding or stitching.

4. The apparatus of claim 1, further comprising a heel counter or toe box as part of the support structure to prevent over-expansion of the bladder.

5. The apparatus of claim 1, wherein the air bladder includes a cavity that houses at least part of the thermal element.

6. The apparatus of claim 1, wherein the control unit is embedded within a heel clip and includes a printed circuit board, a power regulator, a pump, solenoid valves, and a wireless communication component.

7. The apparatus of claim 1, wherein the power source is disposed within a midsole cavity and comprises a rechargeable battery connected via a concealed tunnel.

8. The apparatus of claim 1, further comprising a pressure sensor configured to detect user movement or therapeutic pressure and automatically adjust inflation levels.

9. The apparatus of claim 1, further comprising a modular pod detachably coupled to the apparatus, housing the control unit and the power source.

10. The apparatus of claim 1, wherein the wearable device is a shoe, sandal, slipper, jogger, vest, or glove.

11. The apparatus of claim 1, wherein the user interface includes a power button, heat and compression level buttons, and LED indicators showing power levels, heat levels and compression levels.

12. The apparatus of claim 1, wherein the thermal element includes a thermistor and thermal cutoff switch to prevent overheating.

13. The apparatus of claim 1, wherein the air bladder comprises a spandex-based lower structure and a neoprene upper support structure.

14. The apparatus of claim 1, wherein multiple zones of heat and compression are independently controllable within a single wearable device.

15. The apparatus of claim 1, wherein the thermal element comprises a pair of resistance wires arranged in a symmetrical maze-like pattern between two fabric layers.

16. The apparatus of claim 1, wherein the thermal element is equipped with a heat spreader for improving thermal distribution across the contact area.

17. A method for delivering targeted heat and compression therapy to a user's foot, the method comprising:
applying a wearable device comprising a thermal element and an inflatable bladder to the foot;
selectively activating the thermal element to apply heat to a target region of the foot;
selectively inflating the bladder to apply pressure to a same or overlapping region;
dynamically adjusting the heat or pressure in response to user inputs or sensor feedback; and
deflating the bladder or lowering heat upon detecting user motion or a preset condition.

18. A sandal for delivering heat and compression therapy to a user's foot, comprising:
a sole including a midsole and an outsole, the midsole defining a housing for a power source and control electronics;
an upper strap configured to secure the sandal to a user's foot, the upper strap comprising:
at least one thermal element configured to apply heat to a targeted portion of the foot, and
at least one air bladder configured to apply compression to the same or overlapping portion of the foot;
a control unit operatively connected to the thermal element and the air bladder, the control unit comprising at least one pump for inflating the air bladder; and
a power source configured to power the thermal element and control unit, wherein the power source is housed within the midsole,
wherein the thermal element and air bladder are integrated into the upper strap such that therapeutic heat and pressure are applied directly to a dorsal surface of the foot.

19. The sandal of claim 18, wherein the control unit further comprises a temperature sensor and a pressure sensor for regulating heat and compression levels, and a user interface comprising one or more buttons and indicators integrated into the sandal.

20. The sandal of claim 18, wherein the upper strap includes a removably attached pod, the pod housing the control unit and power source.
